(19) United States
(12) Patent Application Publication
(10) Pub. No.: US 2023/0272419 A1
(43) Pub. Date: Aug. 31, 2023

(54) CANINE AND FELINE INDUCIBLE EXPRESSION CONSTRUCTS FOR GENE THERAPY APPLICATIONS

(71) Applicant: The Trustees of the University of Pennsylvania, Philadelphia, PA (US)

(72) Inventors: James M. Wilson, Philadelphia, PA (US); Christian Hinderer, Baltimore, MD (US); Makoto Horiuchi, Wallingford, PA (US)

(73) Assignee: The Trustees of the University of Pennsylvania, Philadelphia, PA (US)

(21) Appl. No.: 18/006,900

(22) PCT Filed: Jul. 26, 2021
(86) PCT No.: PCT/US2021/043219

§ 371 Date: Jan. 26, 2023

Related U.S. Application Data
(60) Provisional application No. 63/056,985, filed on Jul. 27, 2020.

(51) Int. Cl. C12N 15/86; C07K 14/47

(52) U.S. Cl. CPC C12N 15/86; C07K 14/4702; C07K 2319/81; C12N 2750/14143; C07K 2319/71; C07K 2319/33; C12N 2840/203

(57) ABSTRACT

Provided herein are nucleic acid molecules, vectors, and recombinant AAV comprising an inducible gene expression system. The system includes a transgene encoding a gene product operably linked to expression control sequences comprising a promoter; an activation domain comprising a canine or feline transactivation domain and a FKBP12-rapamycin binding (FRB) domain of canine or feline FKBP12-rapamycin-associated protein (FRAP); a DNA binding domain comprising a zinc finger homeodomain (ZFHD) and one, two or three FK506 binding protein domain (FKBP) subunit genes; and at least 8 copies of the binding site for ZFHD (8XZFHD) followed by a minimal IL2 promoter. The presence of an effective amount of a rapamycin or a rapalog induces expression of the transgene in a host cell.

### BACKGROUND

A central challenge for gene therapy is the difficulty of modulating expression of the transgene in vivo. Virtually all pre-clinical and clinical applications of gene therapy have used vectors that express the transgene from a constitutive promoter, which means it is active at a fixed level for as long as the vector genome persists. However, many diseases that are amenable to gene therapy may need to have expression of the transgene regulated. Several systems have been described that are based on the general principle of placing a gene of interest under the control of a drug-inducible engineered transcription factor in order to positively induce gene expression (Clackson et at., 1997, Curr Opin Chem BioI, 1 (2): 210-8; Rossi et at., Curr Opin Biotechnol, 1998.9(5): p. 451-6). Systems have previously been developed that allow the expression of the transgene to be induced by administration of a small molecule drug, such as rapamycin, or a rapalog.

Rapalog-regulated gene expression systems are described for example in U.S. Pat. Nos. 6,015,709; 6,117,680; 6,133,456; 6,150,527; 6,187,757; 6,306,649; 6,479,653 and 6,649,595. Two major systems, which employ the ARIAD® technology, include a system based on homodimerization and a system based on heterodimerization (Rivera et al., 1996, Nature Med, 2(9):1028-1032; Ye et al., 2000, Science 283: 88-91; Rivera et al., PNAS, Vol. 96(15): 8657-8662, 1999).

As summarized in J. Naidoo and D. Young, Neurology Research International, Vol 2012, Article ID 595410, 10 pages, (2011), a rapamycin-regulated gene regulation system relies on the interaction between two transcription factors, one incorporating a DNA-binding domain and the other a DNA activation domain. Each of the transcription factors also contains a heterologous ligand-binding domain that enables interaction in the presence of the dimerizing drug rapamycin to drive transgene expression. DNA binding is facilitated through the human CMV promoter driven production of a zinc finger homeodomain-1 (ZFHD1) DNA-binding domain fused to three copies of the FK-binding protein (FKBP). Transgene expression is achieved in the presence of rapamycin, which induces dimerization of this DNA-binding protein with a fusion protein consisting of the FKBP-rapamycin-associated protein 1 (FRAP) fused to the NFκB p65 activation domain.

While these systems would be beneficial in the therapy of non-human animals, all of the systems that have been reported are based on human sequences. Therefore, what is needed are regulatable systems suitable for non-human therapies.

### SUMMARY

Provided herein are nucleic acid molecules, vectors, and recombinant AAV comprising an inducible gene expression system.

In a first aspect, a nucleic acid molecule comprising the inducible gene expression system is provided. The system includes a transgene encoding a gene product operably linked to expression control sequences comprising a promoter; an activation domain comprising a canine or feline transactivation domain and a FKBP12-rapamycin binding (FRB) domain of canine or feline FKBP12-rapamycin-associated protein (FRAP); a DNA binding domain comprising a zinc finger homeodomain (ZFHD) and one, two or three FK506 binding protein domain (FKBP) subunit genes; and at least 8 copies of the binding site for ZFHD (8XZFHD) followed by a minimal IL2 promoter. The presence of an effective amount of a rapamycin or a rapalog induces expression of the transgene in a host cell.

In another aspect, a nucleic acid molecule is provided. The molecule includes a promoter; an activation domain comprising a canine or feline p65 transactivation domain and a FKBP12-rapamycin binding (FRB) domain of canine or feline FKBP12-rapamycin-associated protein (FRAP); a DNA binding domain comprising a zinc finger homeodomain (ZFHD) and three FK506 binding protein domain (FKBP) subunit genes; 8 copies of the binding site for ZFHD, and a coding sequence for a therapeutic product.

In another aspect, a viral vector comprising a nucleic acid molecule comprising the inducible gene expression system as described herein is provided.

In another aspect, a recombinant AAV (rAAV) comprising a nucleic acid molecule comprising the inducible gene expression system as described herein is provided.

In another aspect, a therapeutic regimen for regulating the dose of a therapeutic product is provided. The regimen includes administering a rAAV composition as described herein to a subject in need thereof, and delivering an effective amount of a rapamycin or a rapalog to induce expression of the therapeutic product in a host cell of the subject. In one embodiment, the subject is a canine or feline.

Other aspects and advantages of the invention will be readily apparent from the following detailed description of the invention.

### BRIEF DESCRIPTION OF THE DRAWINGS

FIG. 1 is a plasmid map showing cTF.rhEpo.3w.rBG (canine inducible cassette).

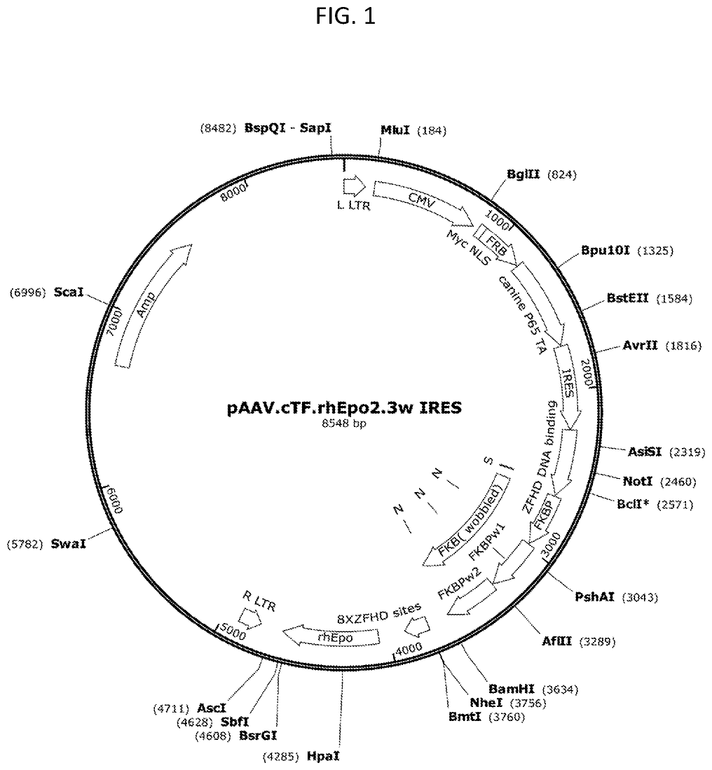

FIG. 2 is a plasmid map showing fTF.rhEpo.3w.rBG (feline inducible cassette).

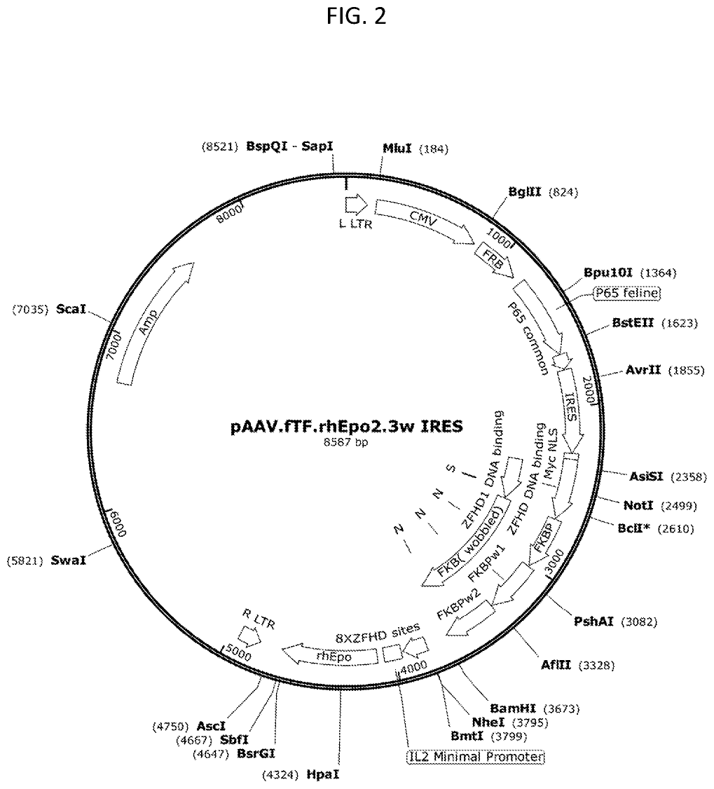

FIG. 3 is a graph showing in vitro expression of rhEPO. HEK293 cells were transfected with hTF.rhEpo.3w.rBG (human inducible cassette), cTF.rhEpo.3w.rBG (canine inducible cassette), or fTF.rhEpo.3w.rBG (feline inducible cassette) expressing rhesus macaque Epo (rhEpo). Cells were treated with 0 nM, 4 nM, or 40 nM of rapamycin a day after transfection. Culture supernatants were collected and rhEpo was measured at 48 h after rapamycin treatment.

FIG. 4 provides amino acid sequences for feline inducible constructs, feFRB-p65 (SEQ ID NO: 36) and feZFHD1-3xFKBP (SEQ ID NO: 37), and canine inducible constructs, caFRB-p65 (SEQ ID NO: 38) and caZFHD1-3xFKBP (SEQ ID NO: 39).

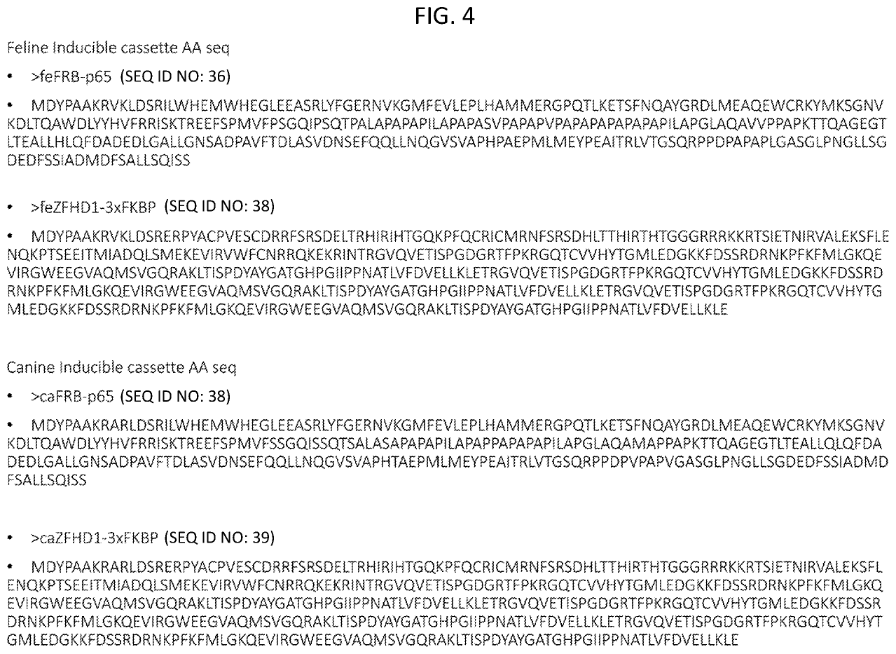

### DETAILED DESCRIPTION

The nucleic acids, vectors and viruses described herein are designed to allow a gene therapy to be administered once to a subject, after which the transgene is turned on by administering an oral inducer. This allows periodic oral medications to replace frequent injections for biologic drugs. In one embodiment, the subject is a canine. In another embodiment, the subject is a feline.

While all previously available systems are based on proteins from humans or other species, provided herein are AAV-packaged constructs comprised entirely of canine and feline amino acid sequences that have better safety profiles and decreased immunogenicity in veterinary applications.

It is to be noted that the term “a” or “an” refers to one or more. As such, the terms “a” (or “an”), “one or more,” and “at least one” are used interchangeably herein.

While various embodiments in the specification are presented using “comprising” language, under other circumstances, a related embodiment is also intended to be interpreted and described using “consisting of” or “consisting essentially of” language. The words “comprise”, “comprises”, and “comprising” are to be interpreted inclusively rather than exclusively. The words “consist”, “consisting”, and its variants, are to be interpreted exclusively, rather than inclusively.

As used herein, the term “about” means a variability of 10% from the reference given, unless otherwise specified.

The term “regulation” or variations thereof as used herein refers to the ability of a compound of formula (I) to inhibit one or more components of a biological pathway.

A “subject” is a mammal, e.g., a mouse, rat, guinea pig, canine, feline, horse, cow, pig, or non-human primate, such as a monkey, chimpanzee, baboon or gorilla. In one embodiment, the subject is a feline. In another embodiment, the subject is a canine.

As used herein, “disease”, “disorder” and “condition” are used interchangeably, to indicate an abnormal state in a subject.

Unless defined otherwise in this specification, technical and scientific terms used herein have the same meaning as commonly understood by one of ordinary skill in the art and by reference to published texts, which provide one skilled in the art with a general guide to many of the terms used in the present application.

The following components are described, in some instances, with respect to a nucleic acid molecule. It is to be understood that where a nucleic acid molecule (or vector or virus (e.g., rAAV)) is recited, in another embodiment, a vector or virus (or nucleic acid molecule) is encompassed herein.

### Components

Provided herein are nucleic acid molecules, vectors, and viruses which include an improved rapamycin/rapalog regulatable expression system. As provided herein, the dose of the transgene product delivered via the vectors provided herein is regulated (controlled) by the regulating or inducing agent (small molecule), also sometimes called a “dimerizer”, delivered to the subject. Thus, delivery of the rapamycin or rapalog brings together the two components required to activate expression of the transgene.

The inducible gene regulation/expression system provided herein contains at least the following components: a promoter operably linked to a transgene encoding a gene product of interest, an activation domain, DNA binding domain, and zinc finger homeodomain binding site(s). In other embodiments, additional components may be included in the expression system, as further described herein.

The nucleic acid molecule comprises an activation domain, which is preferably located upstream of the DNA binding domain. The system described herein has been designed to utilize canine or feline sequences in the activation domain. In one embodiment, the activation domain is a fusion of the carboxy terminus from the p65 subunit of NF-kappa B and FKBP12-rapamycin binding (FRB) domain of FKBP12-rapamycin-associated protein (FRAP). In one embodiment, the FRB domain is a canine sequence. In another embodiment, the FRB domain is a feline sequence.

In one embodiment, the activation domain is a FKBP12-rapamycin binding (FRB) domain of feline FKBP12-rapamycin-associated protein (FRAP) fused to a carboxy terminus from the p65 subunit of NF-kappa B from a feline. In another embodiment, the activation domain is a FKBP12-rapamycin binding (FRB) domain of a canine FKBP12-rapamycin-associated protein (FRAP) fused to a carboxy terminus from the p65 subunit of NF-kappa B from a canine.

In one embodiment, the FRB domain has a sequence of SEQ ID NO: 1 (canine):

atcctctggcatgagatgtggcatgaaggcctggaagaggcatctcgtt
tgtactttggggaaaggaacgtgaaaggcatgtttgaggtgctggagcc
cttgcatgctatgatggaacggggcccccagactctgaaggaaacatcc
tttaatcaggcctatggtcgagatttaatggaggcccaagagtggtgca
ggaagtacatgaaatcagggaacgtcaaggacctcacccaagcctggga
cctctattatcacgtgttccgacgaatctcaaag.

In one embodiment, the FRB domain has a sequence of SEQ ID NO: 2 (feline):

atggactatcctgctgccaagagggtcaagttggactctagaatcctct
ggcatgagatgtggcatgaaggcctggaagaggcatctcgtttgtactt
tggggaaaggaacgtgaaaggcatgtttgaggtgctggagcccttgcat
gctatgatggaacggggcccccagactctgaaggaaacatcctttaatc
aggcctatggtcgagatttaatggaggcccaagagtggtgcaggaagta
catgaaatcagggaacgtcaaggacctcacccaa.

For comparison, the human FRB domain sequence is shown in SEQ ID NO: 3 (human):

atggactatcctgctgccaagagggtcaagttggactctagaatcctct
ggcatgagatgtggcatgaaggcctggaagaggcatctcgtttgtactt
tggggaaaggaacgtgaaaggcatgtttgaggtgctggagcccttgcat
gctatgatggaacggggcccccagactctgaaggaaacatcctttaatc
aggcctatggtcgagatttaatggaggcccaagagtggtgcaggaagta
catgaaatcagggaatgtcaaggacctcctccaa.

In other embodiments, the FRB domain has a sequence sharing at least 90%, 95%, 96%, 96%, 98%, or 99% identity with SEQ ID NO: 1. In another embodiment, the FRB domain has a sequence sharing at least 90%, 95%, 96%, 96%, 98%, or 99% identity with SEQ ID NO: 2.

In one embodiment, the p65 subunit of NF-kappa B is a canine sequence. In another embodiment, the p65 subunit of NF-kappa B is a feline sequence.

In one embodiment, the p65 subunit has a sequence of SEQ ID NO: 4 (canine):

gaggaattcagccccatggtgttctctagcggccagatcagctctcaga
caagcgcccttgcttctgcccctgctcctgctccaattctggctccagc
tcctcctgcaccagctccagcacctattctggcacctggactggctcag
gctatggctccaccagcaccaaagacaacacaggccggcgagggaacac
tgacagaagctctgctgcagctccagttcgacgccgacgaggatctggg
agccctgctgggcaattctgctgatcctgccgtgttcaccgatctggcc
agcgtggacaatagcgagttccagcagctcctgaaccagggcgttagtg
tggcccctcatacagccgagcctatgctgatggaataccccgaggctat
caccagactggtcaccggctctcaaagacctccagatcctgtgcctgct
ccagtgggagcatctgggctccccaatggcctcctttcaggagatgaag
acttctcctccattgcggacatggacttctcagccctgctgagtcagat
cagctcc

In one embodiment, the p65 subunit has a sequence of SEQ ID NO: 5 (feline):

gaggaattcagccccatggtgttccccagcggacagatcccatctcaga
caccagctctggctccagctcctgctcctattcttgctcctgcaccagc
ctctgtgcctgctccagcacctgttccggcaccagcaccagctccggct
ccggctccagcaccaatacttgctccaggactggctcaggctgtggtgc
cgccagctcctaaaacaacacaagccggcgagggcacactgacagaagc
tctgctgcatctgcagttcgacgccgacgaggatctgggagccctgctg
ggcaattctgctgatcctgccgtgttcaccgatctggccagcgtggaca
atagcgagttccagcagctcctgaaccagggcgttagtgtggctcctca
tcctgccgagcctatgctgatggaataccccgaggctatcaccagactg
gtcaccggctctcaaagacctccagatccagcaccggcaccactgggag
catct

For comparison, the human p65 subunit sequence is shown in SEQ ID NO: 6 (human):

gatgagtttcccaccatggtgtttccttctgggcagatcagccaggcct
cggccttggccccggcccctccccaagtcctgccccaggctccagcccc
tgcccctgctccagccatggtatcagctctggcccaggccccagcccct
gtcccagtcctagccccaggccctcctcaggctgtggccccacctgccc
ccaagcccacccaggctggggaaggaacgctgtcagaggccctgctgca
gctgcagtttgatgatgaagacctgggggccttgcttggcaacagcaca
gacccagctgtgttcacagacctggcatccgtcgacaactccgagtttc
agcagctgctgaaccagggcatacctgtggccccccacacaactgagcc
catgctgatggagtaccctgaggctataactcgcctagtgacaggggcc
cagaggccccccgacccagctcctgctccactgggggccccggggctcc
ccaatggcctcctttcaggagatgaagacttctcctccattgcggacat
ggacttctcagccctgctgagtcagatcagctcc

In other embodiments, the p65 subunit has a sequence sharing at least 90%, 95%, 96%, 96%, 98%, or 99% identity with SEQ ID NO: 4. In another embodiment, the p65 subunit has a sequence sharing at least 90%, 95%, 96%, 96%, 98%, or 99% identity with SEQ ID NO: 5.

The DNA binding domain is composed of a DNA-binding fusion of zinc finger homeodomain 1 joined to up to three copies of FK506 binding protein (FKBP). In the presence of an inducing agent, e.g., a rapalog such as rapamycin, the DNA binding domain and activation domain are dimerized through interaction of their FKBP and FRB domains, leading to transcription activation of the transgene.

In one embodiment, the inducible expression system is contained in the same vector as the promoter and coding sequence for the therapeutic product. In another embodiment, the inducible expression system is contained in two or more vectors. The system further comprises the inducible promoter upstream of the coding sequence for the therapeutic product of interest.

In one embodiment, the zinc finger homeodomain 1 has the sequence of SEQ ID NO: 16. In another embodiment, the zinc finger homeodomain 1 has a sequence sharing at least 90%, 95%, 96%, 96%, 98%, or 99% identity with SEQ ID NO: 30.

The nucleic acid molecule is designed to have one, two or three copies of the FKBP sequence. These are termed herein FKBP subunits. In one embodiment, the subunits are designed to express the same protein, but to have nucleic acids which are divergent from one another in order to minimize recombination. Examples of suitable FKBP sequences are provided herein. In one embodiment, the selected FKBP subunits are less than about 85% identical to each other, i.e., at least about 15% divergent. In another embodiment, the FKBP subunit nucleic acid sequences are identical.

Examples of suitable FKBP subunit sequences are provided herein. In certain embodiments, the FKBP subunits are about 60% to about 80% identical to the wild-type FKBP coding sequence. However, other suitable sequences may be designed.

In one embodiment, a FKBP subunit has a sequence of SEQ ID NO: 7 (FKBP):

ggagtgcaggtggaaaccatctccccaggagacgggcgcaccttcccca
agcgcggccagacctgcgtggtgcactacaccgggatgcttgaagatgg
aaagaaatttgattcctcccgggacagaaacaagccctttaagtttatg
ctaggcaagcaggaggtgatccgaggctgggaagaaggggttgcccaga
tgagtgtgggtcagagagccaaactgactatatctccagattatgccta
tggtgccactgggcacccaggcatcatcccaccaaacgccactctcgtc
ttcgatgtggagcttctaaaactggaa

In one embodiment, a FKBP subunit has a sequence of SEQ ID NO: 8 (FKBPw1):

ggcgttcaggtggaaaccatcagtccaggggatggccgaacttttccaa
agagagggcagacttgcgtcgtgcattatactggtatgctggaggatgg
gaaaaagttcgactcttccagagatcggaacaaaccattcaaattcatg
ctcgggaaacaggaagttatccgcggatgggaggagggcgtggcccaga
tgtccgtgggccagcgcgccaagctaaccatctccccagactacgccta
cggagccaccggacaccccggtatcatacccccaaacgccacccttgtg
tttgacgtggaactgcttaagctagag

In one embodiment, a FKBP subunit has a sequence of SEQ ID NO: 9 (FKBPw2):

ggcgtgcaggtcgagaccatcagccccggcgacggccgcacctttccca
agagaggccagacttgcgtggtccactacaccggcatgctggaggacgg
caagaagttcgacagcagccgcgaccgcaacaagcccttcaagttcatg
ctgggcaaacaggaagtgatccgcggctgggaggaaggcgtggctcaga
tgagcgtggggcagcgggccaagctgaccatcagccccgactatgccta
cggcgccaccggccaccccggcatcatcccccccaacgccaccctcgtg
ttcgacgtggagctgctgaagctggagtga

(FKBP (full-all subunits)): SEQ ID NO: 10

ggagtgcaggtggaaaccatctccccaggagacgggcgcaccttcccca
agcgcggccagacctgcgtggtgcactacaccgggatgcttgaagatgg
aaagaaatttgattcctcccgggacagaaacaagccctttaagtttatg
ctaggcaagcaggaggtgatccgaggctgggaagaaggggttgcccaga
tgagtgtgggtcagagagccaaactgactatatctccagattatgccta
tggtgccactgggcacccaggcatcatcccaccaaacgccactctcgtc
ttcgatgtggagcttctaaaactggaaactagaggcgttcaggtggaaa
ccatcagtccaggggatggccgaacttttccaaagagagggcagacttg
cgtcgtgcattatactggtatgctggaggatgggaaaaagttcgactct
tccagagatcggaacaaaccattcaaattcatgctcgggaaacaggaag
ttatccgcggatgggaggagggcgtggcccagatgtccgtgggccagcg
cgccaagctaaccatctccccagactacgcctacggagccaccggacac
cccggtatcatacccccaaacgccacccttgtgtttgacgtggaactgc
ttaagctagagactagaggcgtgcaggtcgagaccatcagccccggcga
cggccgcacctttcccaagagaggccagacttgcgtggtccactacacc
ggcatgctggaggacggcaagaagttcgacagcagccgcgaccgcaaca
agcccttcaagttcatgctgggcaaacaggaagtgatccgcggctggga
ggaaggcgtggctcagatgagcgtggggcagcgggccaagctgaccatc
agccccgactatgcctacggcgccaccggccaccccggcatcatccccc
ccaacgccaccctcgtgttcgacgtggagctgctgaagctggagtga

Optionally, one of the subunit sequences may be a wild-type FKBP sequence. In one embodiment, the wild-type FKBP sequence is located upstream of an engineered FKBP subunit sequence. In another embodiment, the wild-type FKBP sequence is located downstream of an engineered FKBP subunit sequence. In still another embodiment, the wild-type FKBP sequence is sandwiched between two different engineered FKBP subunit sequences. In another embodiment, the wild-type FKBP subunit sequence is not used in the composition of the invention.

In one embodiment, the nucleic acid molecule comprises SEQ ID NO: 7, SEQ ID NO: 8, and SEQ ID NO: 9.

The nucleic acid molecule further comprises zinc finger homeodomain binding sites. The nucleic acid molecule contains at least 1, 2, 3, 4, 5, 6, 7, 8, 9, 10, 11, or 12 binding sites for ZFHD. In one embodiment, the nucleic acid molecule contains 8 (eight) zinc finger homeodomains binding site (binding partners) (8XZFHD). However, the invention encompasses nucleic acid molecules having from two to about twelve copies of the zinc finger binding site. In one embodiment, the nucleic acid encompasses 8, 9, 10, 11, or 12 copies of the zinc finger binding site. In one embodiment, the ZFHD binding site comprises SEQ ID NO: 31. In another embodiment, the ZFHD binding site comprises one or more of SEQ ID NO: 32, 33, 34, or 35. The ZFHD binding site may contain more than one copy of any of SEQ ID NO: 32, 33, 34, or 35.

In some embodiments, there is a minimal IL2 promoter downstream of the zinc finger homeodomain binding sites. In one embodiment, the IL2 promoter has the sequence of SEQ ID NO: 11:

aacattttgacacccccataatatttttccagaattaacagtataaatt
gcatctcttgttcaagagttccctatcactctctttaatcactactcac
agtaacctcaactcctgccacaa.

In one embodiment, the IL2 promoter has the sequence of SEQ ID NO: 12:

aacattttgacacccccataatatttttccagaattaacagtataaatt
gcatctcttgttcaagagttccctatcactctctttaatcactactcac
agtaacctc

In one embodiment, there is a linker between the transactivation domain and DNA binding domain, which linker may be an F2A or an IRES. In one embodiment, the linker is an IRES.

As provided herein, the dose of the transgene product delivered via the vectors is regulated (controlled) by the regulating or inducing agent (small molecule), also sometimes called a “dimerizer”, delivered to the subject. In one embodiment, the inducing agent is rapamycin or a rapalog.

A “rapamycin” is a macrolide antibiotic produced by Streptomyces hygroscopicus which binds to a FK506-binding protein, FKBP, with high affinity to form a rapamycin:FKBP complex. The rapamycin:FKBP complex binds with high affinity to the large cellular protein, FRAP, to form an FKBP/rapamycin complex with FRAP. Rapamycin acts as a dimerizer or adapter to join FKBP to FRAP. Rapamycin is also known as sirolimus.

As used herein, the term “rapalog” is meant to include structural variants of rapamycin including analogs, homologs, derivatives and other compounds related structurally to rapamycin. Rapalogs are designed to bind to FRAPL, a mutant of FRAP, but not to wild type FRAP. Such structural variants include modifications such as demethylation, elimination or replacement of the methoxy at C7, C42 and/or C29; elimination, derivatization or replacement of the hydroxy at C13, C43 and/or C28; reduction, elimination or derivatization of the ketone at C14, C24 and/or C30; replacement of the 6-membered pipecolate ring with a 5-membered prolyl ring; and alternative substitution on the cyclohexyl ring or replacement of the cyclohexyl ring with a substituted cyclopentyl ring. See, e.g., U.S. Pat. Nos. 6,187,757; 5,525,610; 5,310,903 and 5,362,718, expressly incorporated by reference herein. Exemplary rapalogs include, AP22594 (28-epi-rapamycin) which is particularly suitable because it provides the inducing activity of rapamycin with significantly lower immunosuppressive properties. This compound may be synthesized by mixing sirolimus (rapamycin) with methylenechlorside in the presence of Ti(OiPr)₄. After a 60-minute reaction, crude product is dissolved in methanol and recrystallized from the methanol/water mixture. Typical final yield after purification is about 50%. However, other suitable methods may be used. Still other exemplary rapalogs include, e.g., temsirolimus, everolimus, ABT578, AP23573 and biolimus. AP26113 (Ariad), AP1510 (Amara, J. F., et al., 1997, Proc Natl Acad Sci USA, 94(20): 10618-23) AP22660, AP22594, AP21370, AP22594, AP23054, AP1855, AP1856, AP1701, AP1861, AP1692 and AP1889, with designed ‘bumps’ that minimize interactions with endogenous FKBP.

A “rapamycin-regulated promoter” refers to a promoter the activity of which is regulated by the presence or absence of rapamycin. More particularly, control may be more finely regulated than “on” and “off” and the level of transcription may be controlled by the concentrations or doses of rapamycin provided. As provided herein, the promoter that is operatively linked to the transgene is a regulated promoter when included in the inducible gene expression system described herein. Rapamycin inducible promoters are known in the art. See, e.g., WO2007126798, WO2001098507, which are incorporated herein by reference.

In one embodiment, the promoter is a cytomegalovirus (CMV) promoter. In one embodiment, the promoter is a CMV promoter having the sequence of SEQ ID NO: 13:

acgcgttcgagctcgccccgttacataacttacggtaaatggcccgcct
ggctgaccgcccaacgacccccgcccattgacgtcaataatgacgtatg
ttcccatagtaacgccaatagggactttccattgacgtcaatgggtgga
gtatttacggtaaactgcccacttggcagtacatcaagtgtatcatatg
ccaagtacgccccctattgacgtcaatgacggtaaatggcccgcctggc
attatgcccagtacatgaccttatgggactttcctacttggcagtacat
ctacgtattagtcatcgctattaccatggtgatgcggttttggcagtac
atcaatgggcgtggatagcggtttgactcacggggatttccaagtctcc
accccattgacgtcaatgggagtttgttttggcaccaaaatcaacggga
ctttccaaaatgtcgtaacaactccgccccattgacgcaaatgggcggt
aggcgtgtacggtgggaggtctatataagcagagctcgtttagtgaacc
gtcagatcgcctggagacgccatccacgctgttttgacctccatagaag
acaccgggaccgatccagcctccgggggatcttggtggcgtgaaactcc
cg.

Examples of suitable promoters include, without limitation, the retroviral Rous sarcoma virus (RSV) LTR promoter (optionally with the RSV enhancer), the cytomegalovirus (CMV) promoter (optionally with the CMV enhancer) [see, e.g., Boshart et al, Cell, 41:521-530 (1985)], the SV40 promoter, the dihydrofolate reductase promoter, the β-actin promoter, the phosphoglycerol kinase (PGK) promoter, and the EF1 promoter [Invitrogen]. Alternatively, a tissue-specific promoter may be selected. For instance, if expression in skeletal muscle is desired, a promoter active in muscle should be used. These include the promoters from genes encoding skeletal β-actin, myosin light chain 2A, dystrophin, muscle creatine kinase, as well as synthetic muscle promoters with activities higher than naturally-occurring promoters (see Li et al, Nat. Biotech., 17:241-245 (1999)). Examples of promoters that are tissue-specific are known for liver (albumin, Miyatake et al, J. Virol, 71:5124-32 (1997); hepatitis B virus core promoter (Sandig et al., Gene Ther., 3:1002-9 (1996)); alpha-fetoprotein (AFP, Arbuthnot et al., Hum. Gene Ther., 7:1503-14 (1996)), bone osteocalcin (Stein et al., Mol. Biol. Rep., 24:185-96 (1997)); bone sialoprotein (Chen et al., J. Bone Miner. Res., 11:654-64 (1996)), lymphocytes (CD2, Hansal et al, J. Immunol., 161:1063-8 (1998); therapeutic product heavy chain; T cell receptor chain, neuronal such as neuron-specific enolase (NSE) promoter (Andersen et al, Cell. Mol. Neurobiol., 13:503-15 (1993)), neurofilament light-chain gene (Piccioli et al., Proc. Natl. Acad. Sci. USA, 88:5611-5 (1991)), and the neuron-specific vgf gene (Piccioli et al., Neuron, 15:373-84 (1995)), among others. Promoters may include a retinal pigmented epithelium (RPE) promoter or a photoreceptor promoter which may be derived from any species.

In certain embodiments, the promoter is the native promoter for the gene to be expressed. In still other embodiments, the promoter is the RPGR proximal promoter (Shu et al, IOVS, May 2012, which is incorporated by reference herein). Other useful promoters include, without limitation, the rod opsin promoter, the red-green opsin promoter, the blue opsin promoter, the cGMP-β-phosphodiesterase promoter, the mouse opsin promoter (Beltran et al 2010 cited above), the rhodopsin promoter (Mussolino et al, Gene Ther, July 2011, 18(7):637-45); the alpha-subunit of cone transducin (Morrissey et al, BMC Dev, Biol, January 2011, 11:3); beta phosphodiesterase (PDE) promoter; the retinitis pigmentosa (RP1) promoter (Nicord et al, J. Gene Med, December 2007, 9(12):1015-23); the NXNL2/NXNL1 promoter (Lambard et al, PLoS One, October 2010, 5(10):e13025), the RPE65 promoter; the retinal degeneration slow/peripherin 2 (Rds/perph2) promoter (Cai et al, Exp Eye Res. 2010 August; 91(2):186-94); and the VMD2 promoter (Kachi et al, Human Gene Therapy, 2009 (20:31-9)). Examples of photoreceptor specific promoters include, without limitation, the rod opsin promoter, the red-green opsin promoter, the blue opsin promoter, the inter photoreceptor binding protein (IRBP) promoter and the cGMP-β-phosphodiesterase promoter.

The nucleic acid further contains a coding sequence for a gene product under the control of the regulatable promoter. The transgene is a nucleic acid sequence, heterologous to the vector sequences flanking the transgene, which encodes a polypeptide, protein, or other product, of interest. The nucleic acid coding sequence is operatively linked to regulatory components in a manner which permits transgene transcription, translation, and/or expression in a target cell. The heterologous nucleic acid sequence (transgene) can be derived from any organism. The nucleic acid molecule, vector or rAAV may comprise one or more transgenes.

In certain embodiments, provided herein is a nucleic acid molecule, vector, or rAAV that includes a transgene comprising a sequence encoding erythropoietin (EPO). In certain embodiments, the transgene encodes a canine or feline EPO gene. Such recombinant vectors are suitable, for example, for use in a regimen for treating chronic kidney disease and other conditions in a subject characterized by a decrease in the amount of circulating red blood cells.

In certain embodiments, provided herein is a nucleic acid molecule, vector, or rAAV that includes a transgene comprising a sequence encoding an anti-nerve growth factor (NGF) antibody. In certain embodiments, the transgene encodes a canine or feline anti-NGF antibody. Such recombinant vectors are suitable, for example, for use in a regimen for treating osteoarthritis pain in a subject. See, e.g., Gearing et al, In Vitro and In Vivo Characterization of a Fully Felinized Therapeutic Anti-Nerve Growth Factor Monoclonal Antibody for the Treatment of Pain in Cats, J Vet Intern Med. 2016 July;30(4):1129-37. doi: 10.1111/jvim.13985. Epub 2016 Jun. 15; and Gearing et al, A Fully Caninised anti-NGF Monoclonal Antibody for Pain Relief in Dogs, BMC Vet Res. 2013 Nov. 9; 9:226. doi: 10.1186/1746-6148-9-226, which are incorporated herein by reference

In certain embodiments, provided herein is a nucleic acid molecule, vector, or rAAV that includes a transgene comprising a sequence encoding an anti-CD20 antibody. In certain embodiments, the transgene encodes a canine or feline anti-CD20 antibody. Such recombinant vectors are suitable, for example, for use in a regimen for treating B cell lymphoma in a subject.

In certain embodiments, provided herein is a nucleic acid molecule, vector, or rAAV that includes a transgene comprising a sequence encoding an anti-GnRH antibody. In certain embodiments, the transgene encodes a canine or feline anti-GnRH antibody. Such recombinant vectors are suitable, for example, for use in a regimen for sterilization of a feline or canine subject.

In certain embodiments, provided herein is a nucleic acid molecule, vector, or rAAV that includes a transgene comprising a sequence encoding an anti-IL-31 antibody. In certain embodiments, the transgene encodes a canine or feline anti-IL-31 antibody.

In certain embodiments, provided herein is a nucleic acid molecule, vector, or rAAV that includes a transgene comprising a sequence encoding glucagon-like peptide 1 (GLP-1) and analogs thereof. In certain embodiments, the transgene encodes a GLP-1 receptor antagonist targeted to a canine or feline. Such recombinant vectors are suitable, for example, for use in a regimen for treating type II diabetes in a subject.

In certain embodiments, provided herein is a nucleic acid molecule, vector, or rAAV that includes a transgene comprising a sequence encoding Granulocyte-macrophage colony-stimulating factor (GM-CSF). In certain embodiments, the transgene encodes GM-CSF targeted to a canine or feline.

In certain embodiments, provided herein is a nucleic acid molecule, vector, or rAAV that includes a transgene comprising a sequence encoding granulocyte colony-stimulating factor (G-CSF). In certain embodiments, the transgene encodes G-CSF targeted to a canine or feline.

In certain embodiments, provided herein is a nucleic acid molecule, vector, or rAAV that includes a transgene comprising a sequence encoding insulin. In certain embodiments, the transgene encodes a canine or feline insulin. Such recombinant vectors are suitable, for example, for use in a regimen for treating a metabolic disease including type I diabetes or type II diabetes, in a subject.

In certain embodiments, provided herein is a nucleic acid molecule, vector, or rAAV that includes a transgene comprising a sequence encoding an antagonist for IgE, IL-32, or the interleukin-4 receptor alpha (IL-4Rα) subunit of IL-4/IL-13 receptors, including, e.g., antibodies and receptor-IgG fusion proteins. In certain embodiments, the transgene encodes an antagonist for a canine or feline IgE, IL-32, or IL-4Ra subunit. Such recombinant vectors are suitable, for example, for use in a regimen for treating atopic dermatitis in a subject.

The composition of the transgene sequence will depend upon the use to which the resulting vector will be put. For example, one type of transgene sequence includes a reporter sequence, which upon expression produces a detectable signal. Such reporter sequences include, without limitation, DNA sequences encoding β-lactamase, β-galactosidase (LacZ), alkaline phosphatase, thymidine kinase, green fluorescent protein (GFP), enhanced GFP (EGFP), chloramphenicol acetyltransferase (CAT), luciferase, membrane bound proteins including, for example, CD2, CD4, CD8, the influenza hemagglutinin protein, and others well known in the art, to which high affinity antibodies directed thereto exist or can be produced by conventional means, and fusion proteins comprising a membrane bound protein appropriately fused to an antigen tag domain from, among others, hemagglutinin or Myc.

These coding sequences, when associated with regulatory elements which drive their expression, provide signals detectable by conventional means, including enzymatic, radiographic, colorimetric, fluorescence or other spectrographic assays, fluorescent activating cell sorting assays and immunological assays, including enzyme linked immunosorbent assay (ELISA), radioimmunoassay (RIA) and immunohistochemistry. For example, where the marker sequence is the LacZ gene, the presence of the vector carrying the signal is detected by assays for beta-galactosidase activity. Where the transgene is green fluorescent protein or luciferase, the vector carrying the signal may be measured visually by color or light production in a luminometer.

However, desirably, the transgene is a non-marker sequence encoding a product which is useful in biology and medicine, such as proteins, peptides, RNA, enzymes, dominant negative mutants, or catalytic RNAs. Desirable RNA molecules include tRNA, dsRNA, ribosomal RNA, catalytic RNAs, siRNA, small hairpin RNA, trans-splicing RNA, and antisense RNAs. One example of a useful RNA sequence is a sequence which inhibits or extinguishes expression of a targeted nucleic acid sequence in the treated animal. Typically, suitable target sequences include oncologic targets and viral diseases. See, for examples of such targets the oncologic targets and viruses identified below in the section relating to immunogens.

The transgene may be used to correct or ameliorate gene deficiencies, which may include deficiencies in which normal genes are expressed at less than normal levels or deficiencies in which the functional gene product is not expressed. Alternatively, the transgene may provide a product to a cell which is not natively expressed in the cell type or in the host. A preferred type of transgene sequence encodes a therapeutic protein or polypeptide which is expressed in a host cell. The invention further includes using multiple transgenes. In certain situations, a different transgene may be used to encode each subunit of a protein, or to encode different peptides or proteins. This is desirable when the size of the DNA encoding the protein subunit is large, e.g., for an immunoglobulin, the platelet-derived growth factor, or a dystrophin protein. In order for the cell to produce the multi-subunit protein, a cell is infected with the recombinant virus containing each of the different subunits. Alternatively, different subunits of a protein may be encoded by the same transgene. In this case, a single transgene includes the DNA encoding each of the subunits, with the DNA for each subunit separated by an internal ribozyme entry site (IRES). This is desirable when the size of the DNA encoding each of the subunits is small, e.g., the total size of the DNA encoding the subunits and the IRES is less than five kilobases. As an alternative to an IRES, the DNA may be separated by sequences encoding a 2A peptide, which self-cleaves in a post-translational event. See, e.g., M. L. Donnelly, et al, J. Gen. Virol., 78(Pt 1):13-21 (January 1997); Furler, S., et al, Gene Ther., 8(11):864-873 (June 2001); Klump H., et al., Gene Ther., 8(10):811-817 (May 2001). This 2A peptide is significantly smaller than an IRES, making it well suited for use when space is a limiting factor. More often, when the transgene is large, consists of multi-subunits, or two transgenes are co-delivered, rAAV carrying the desired transgene(s) or subunits are co-administered to allow them to concatamerize in vivo to form a single vector genome. In such an embodiment, a first AAV may carry an expression cassette which expresses a single transgene and a second AAV may carry an expression cassette which expresses a different transgene for co-expression in the host cell. However, the selected transgene may encode any biologically active product or other product, e.g., a product desirable for study.

Useful therapeutic products encoded by the transgene include hormones and growth and differentiation factors including, without limitation, insulin, glucagon, glucagon-like peptide 1 (GLP-1), growth hormone (GH), parathyroid hormone (PTH), growth hormone releasing factor (GRF), follicle stimulating hormone (FSH), luteinizing hormone (LH), human chorionic gonadotropin (hCG), vascular endothelial growth factor (VEGF), angiopoietins, angiostatin, granulocyte colony stimulating factor (GCSF), erythropoietin (EPO), connective tissue growth factor (CTGF), basic fibroblast growth factor (bFGF), acidic fibroblast growth factor (aFGF), epidermal growth factor (EGF), transforming growth factor α (TGFα), platelet-derived growth factor (PDGF), insulin growth factors I and II (IGF-I and IGF-II), any one of the transforming growth factor β superfamily, including TGF β, activins, inhibins, or any of the bone morphogenic proteins (BMP) BMPs 1-15, any one of the heregluin/neuregulin/ARIA/neu differentiation factor (NDF) family of growth factors, nerve growth factor (NGF), brain-derived neurotrophic factor (BDNF), neurotrophins NT-3 and NT-4/5, ciliary neurotrophic factor (CNTF), glial cell line derived neurotrophic factor (GDNF), neurturin, agrin, any one of the family of semaphorins/collapsins, netrin-1 and netrin-2, hepatocyte growth factor (HGF), ephrins, noggin, sonic hedgehog and tyrosine hydroxylase.

Other useful transgene products include proteins that regulate the immune system including, without limitation, cytokines and lymphokines such as thrombopoietin (TPO), interleukins (IL) IL-1 through IL-25 (including, IL-2, IL-4, IL-12, and IL-18), monocyte chemoattractant protein, leukemia inhibitory factor, granulocyte-macrophage colony stimulating factor, Fas ligand, tumor necrosis factors α and β, interferons α, β, and γ, stem cell factor, flk-2/flt3 ligand. Gene products produced by the immune system are also useful in the invention. These include, without limitations, immunoglobulins IgG, IgM, IgA, IgD and IgE, chimeric immunoglobulins, caninized antibodies, felinized antibodies, single chain antibodies, T cell receptors, chimeric T cell receptors, single chain T cell receptors, class I and class II MEC molecules, as well as engineered immunoglobulins and MEC molecules. Useful gene products also include complement regulatory proteins such as complement regulatory proteins, membrane cofactor protein (MCP), decay accelerating factor (DAF), CR1, CF2 and CD59.

Still other useful gene products include any one of the receptors for hormones, growth factors, cytokines, lymphokines, regulatory proteins and immune system proteins. The invention encompasses receptors for cholesterol regulation, including the low-density lipoprotein (LDL) receptor, high density lipoprotein (HDL) receptor, the very low density lipoprotein (VLDL) receptor, and the scavenger receptor. The invention also encompasses gene products such as members of the steroid hormone receptor superfamily including glucocorticoid receptors and estrogen receptors, Vitamin D receptors and other nuclear receptors. In addition, useful gene products include transcription factors such as jun, fos, max, mad, serum response factor (SRF), AP-1, AP2, myb, MyoD and myogenin, ETS-box containing proteins, TFE3, E2F, ATF1, ATF2, ATF3, ATF4, ZFS, NFAT, CREB, HNF-4, C/EBP, SP1, CCAAT-box binding proteins, interferon regulation factor (IRF-1), Wilms tumor protein, ETS-binding protein, STAT, GATA-box binding proteins, e.g., GATA-3, and the forkhead family of winged helix proteins.

Other useful gene products include, carbamoyl synthetase I, ornithine transcarbamylase, arginosuccinate synthetase, arginosuccinate lyase, arginase, fumarylacetacetate hydrolase, phenylalanine hydroxylase, alpha-1 antitrypsin, glucose-6-phosphatase, porphobilinogen deaminase, factor VIII, factor IX, cystathione beta-synthase, branched chain ketoacid decarboxylase, albumin, isovaleryl-coA dehydrogenase, propionyl CoA carboxylase, methyl malonyl CoA mutase, glutaryl CoA dehydrogenase, insulin, beta-glucosidase, pyruvate carboxylate, hepatic phosphorylase, phosphorylase kinase, glycine decarboxylase, H-protein, T-protein, a cystic fibrosis transmembrane regulator (CFTR) sequence, and a dystrophin sequence or functional fragment thereof. Still other useful gene products include enzymes such as may be useful in enzyme replacement therapy, which is useful in a variety of conditions resulting from deficient activity of enzyme. For example, enzymes that contain mannose-6-phosphate may be utilized in therapies for lysosomal storage diseases (e.g., a suitable gene includes that encodes β-glucuronidase (GUSB)). In another example, the gene product is ubiquitin protein ligase E3A (UBE3A). Still useful gene products include UDP Glucuronosyltransferase Family 1 Member A1 (UGT1A1).

Other useful gene products include non-naturally occurring polypeptides, such as chimeric or hybrid polypeptides having a non-naturally occurring amino acid sequence containing insertions, deletions or amino acid substitutions. For example, single-chain engineered immunoglobulins could be useful in certain immunocompromised patients. Other types of non-naturally occurring gene sequences include antisense molecules and catalytic nucleic acids, such as ribozymes, which could be used to reduce overexpression of a target.

Reduction and/or modulation of expression of a gene is particularly desirable for treatment of hyperproliferative conditions characterized by hyperproliferating cells, as are cancers and psoriasis. Target polypeptides include those polypeptides which are produced exclusively or at higher levels in hyperproliferative cells as compared to normal cells. Target antigens include polypeptides encoded by oncogenes such as myb, myc, fyn, and the translocation gene bcr/abl, ras, src, P53, neu, trk and EGRF. In addition to oncogene products as target antigens, target polypeptides for anti-cancer treatments and protective regimens include variable regions of antibodies made by B cell lymphomas and variable regions of T cell receptors of T cell lymphomas which, in some embodiments, are also used as target antigens for autoimmune disease. Other tumor-associated polypeptides can be used as target polypeptides such as polypeptides which are found at higher levels in tumor cells including the polypeptide recognized by monoclonal antibody 17-1A and folate binding polypeptides.

Other suitable therapeutic polypeptides and proteins include those which may be useful for treating individuals suffering from autoimmune diseases and disorders by conferring a broad based protective immune response against targets that are associated with autoimmunity including cell receptors and cells which produce self-directed antibodies. T cell mediated autoimmune diseases include Rheumatoid arthritis (RA), multiple sclerosis (MS), Sjögren's syndrome, sarcoidosis, insulin dependent diabetes mellitus (IDDM), autoimmune thyroiditis, reactive arthritis, ankylosing spondylitis, scleroderma, polymyositis, dermatomyositis, psoriasis, vasculitis, Wegener's granulomatosis, Crohn's disease and ulcerative colitis. Each of these diseases is characterized by T cell receptors (TCRs) that bind to endogenous antigens and initiate the inflammatory cascade associated with autoimmune diseases.

Still other useful gene products include those used for treatment of hemophilia, including hemophilia B (including Factor IX) and hemophilia A (including Factor VIII and its variants, such as the light chain and heavy chain of the heterodimer and the B-deleted domain; U.S. Pat. Nos. 6,200,560 and 6,221,349). In some embodiments, the minigene comprises first 57 base pairs of the Factor VIII heavy chain which encodes the 10 amino acid signal sequence, as well as the human growth hormone (hGH) polyadenylation sequence. In alternative embodiments, the minigene further comprises the A1 and A2 domains, as well as 5 amino acids from the N-terminus of the B domain, and/or 85 amino acids of the C-terminus of the B domain, as well as the A3, C1 and C2 domains. In yet other embodiments, the nucleic acids encoding Factor VIII heavy chain and light chain are provided in a single minigene separated by 42 nucleic acids coding for 14 amino acids of the B domain [U.S. Pat. No. 6,200,560].

Further illustrative genes which may be delivered via the rAAV include, without limitation, glucose-6-phosphatase, associated with glycogen storage disease or deficiency type 1A (GSD1), phosphoenolpyruvate-carboxykinase (PEPCK), associated with PEPCK deficiency; cyclin-dependent kinase-like 5 (CDKL5), also known as serine/threonine kinase 9 (STK9) associated with seizures and severe neurodevelopmental impairment; galactose-1 phosphate uridyl transferase, associated with galactosemia; phenylalanine hydroxylase (PAH), associated with phenylketonuria (PKU); gene products associated with Primary Hyperoxaluria Type 1 including Hydroxyacid Oxidase 1 (GO/HAO1) and AGXT, branched chain alpha-ketoacid dehydrogenase, including BCKDH, BCKDH-E2, BAKDH-E1a, and BAKDH-E1b, associated with Maple syrup urine disease; fumarylacetoacetate hydrolase, associated with tyrosinemia type 1; methylmalonyl-CoA mutase, associated with methylmalonic acidemia; medium chain acyl CoA dehydrogenase, associated with medium chain acetyl CoA deficiency; ornithine transcarbamylase (OTC), associated with ornithine transcarbamylase deficiency; argininosuccinic acid synthetase (ASS1), associated with citrullinemia; lecithin-cholesterol acyltransferase (LCAT) deficiency; amethylmalonic acidemia (MMA); NPC1 associated with Niemann-Pick disease, type C1); propionic academia (PA); TTR associated with Transthyretin (TTR)-related Hereditary Amyloidosis; low density lipoprotein receptor (LDLR) protein, associated with familial hypercholesterolemia (FH), LDLR variant, such as those described in WO 2015/164778; PCSK9; ApoE and ApoC proteins, associated with dementia; UDP-glucouronosyltransferase, associated with Crigler-Najjar disease; adenosine deaminase, associated with severe combined immunodeficiency disease; hypoxanthine guanine phosphoribosyl transferase, associated with Gout and Lesch-Nyan syndrome; biotimidase, associated with biotimidase deficiency; alpha-galactosidase A (a-Gal A) associated with Fabry disease); beta-galactosidase (GLB1) associated with GM1 gangliosidosis; ATP7B associated with Wilson's Disease; beta-glucocerebrosidase, associated with Gaucher disease type 2 and 3; peroxisome membrane protein 70 kDa, associated with Zellweger syndrome; arylsulfatase A (ARSA) associated with metachromatic leukodystrophy, galactocerebrosidase (GALC) enzyme associated with Krabbe disease, alpha-glucosidase (GAA) associated with Pompe disease; sphingomyelinase (SMPD1) gene associated with Nieman Pick disease type A; argininosuccsinate synthase associated with adult onset type II citrullinemia (CTLN2); carbamoyl-phosphate synthase 1 (CPS1) associated with urea cycle disorders; survival motor neuron (SMN) protein, associated with spinal muscular atrophy; ceramidase associated with Farber lipogranulomatosis; b-hexosaminidase associated with GM2 gangliosidosis and Tay-Sachs and Sandhoff diseases; aspartylglucosaminidase associated with aspartyl-glucosaminuria; α-fucosidase associated with fucosidosis; α-mannosidase associated with alpha-mannosidosis; porphobilinogen deaminase, associated with acute intermittent porphyria (AIP); alpha-1 antitrypsin for treatment of alpha-1 antitrypsin deficiency (emphysema); erythropoietin for treatment of anemia due to thalassemia or to renal failure; vascular endothelial growth factor, angiopoietin-1, and fibroblast growth factor for the treatment of ischemic diseases; thrombomodulin and tissue factor pathway inhibitor for the treatment of occluded blood vessels as seen in, for example, atherosclerosis, thrombosis, or embolisms; aromatic amino acid decarboxylase (AADC), and tyrosine hydroxylase (TH) for the treatment of Parkinson's disease; the beta adrenergic receptor, anti-sense to, or a mutant form of, phospholamban, the sarco(endo)plasmic reticulum adenosine triphosphatase-2 (SERCA2), and the cardiac adenylyl cyclase for the treatment of congestive heart failure; a tumor suppressor gene such as p53 for the treatment of various cancers; a cytokine such as one of the various interleukins for the treatment of inflammatory and immune disorders and cancers; dystrophin or minidystrophin and utrophin or miniutrophin for the treatment of muscular dystrophies; and, insulin or GLP-1 for the treatment of diabetes.

Alternatively, or in addition, the vectors of the invention may contain a transgene encoding a peptide, polypeptide or protein which induces an immune response to a selected immunogen. For example, immunogens may be selected from a variety of viral families. Example of desirable viral families against which an immune response would be desirable include, the picornavirus family, which includes the genera rhinoviruses, which are responsible for about 50% of cases of the common cold; the genera enteroviruses, which include polioviruses, coxsackieviruses, echoviruses, and human enteroviruses such as hepatitis A virus; and the genera apthoviruses, which are responsible for foot and mouth diseases, primarily in non-human animals. Within the picornavirus family of viruses, target antigens include the VP1, VP2, VP3, VP4, and VPG. Another viral family includes the calcivirus family, which encompasses the Norwalk group of viruses, which are an important causative agent of epidemic gastroenteritis. Still another viral family desirable for use in targeting antigens for inducing immune responses in humans and non-human animals is the togavirus family, which includes the genera alphavirus, which include Sindbis viruses, RossRiver virus, and Venezuelan, Eastern & Western Equine encephalitis, and rubivirus, including Rubella virus. The flaviviridae family includes dengue, yellow fever, Japanese encephalitis, St. Louis encephalitis and tick-borne encephalitis viruses. Other target antigens may be generated from the Hepatitis C or the coronavirus family, which includes a number of non-human viruses such as infectious bronchitis virus (poultry), porcine transmissible gastroenteric virus (pig), porcine hemagglutinating encephalomyelitis virus (pig), feline infectious peritonitis virus (cats), feline enteric coronavirus (cat), canine coronavirus (dog), and other coronaviruses, which may cause the common cold and/or non-A, B or C hepatitis. In one embodiment, the target antigen is from SARS-COV-2. Within the coronavirus family, target antigens include the E1 (also called M or matrix protein), E2 (also called S or Spike protein), E3 (also called HE or hemagglutin-elterose) glycoprotein (not present in all coronaviruses), or N (nucleocapsid). Still other antigens may be targeted against the rhabdovirus family, which includes the genera vesiculovirus (e.g., Vesicular Stomatitis Virus), and the general lyssavirus (e.g., rabies). Within the rhabdovirus family, suitable antigens may be derived from the G protein or the N protein. The family filoviridae, which includes hemorrhagic fever viruses such as Marburg and Ebola virus may be a suitable source of antigens. The paramyxovirus family includes parainfluenza Virus Type 1, parainfluenza Virus Type 3, bovine parainfluenza Virus Type 3, rubulavirus (mumps virus, parainfluenza Virus Type 2, parainfluenza virus Type 4, Newcastle disease virus (chickens), rinderpest, morbillivirus, which includes measles and canine distemper, and pneumovirus, which includes respiratory syncytial virus. The influenza virus is classified within the family orthomyxovirus and is a suitable source of antigen (e.g., the HA protein, the N1 protein). The bunyavirus family includes the genera bunyavirus (California encephalitis, La Crosse), phlebovirus (Rift Valley Fever), hantavirus (puremala is a hemahagin fever virus), nairovirus (Nairobi sheep disease) and various unassigned bungaviruses. The arenavirus family provides a source of antigens against LCM and Lassa fever virus. The reovirus family includes the genera reovirus, rotavirus (which causes acute gastroenteritis in children), orbiviruses, and cultivirus.

The retrovirus family includes the sub-family orthoretrovirinae which encompasses such veterinary diseases as feline leukemia virus, HTLVI and HTLVII, lentiviranae (which includes human immunodeficiency virus (HIV), simian immunodeficiency virus (SIV), feline immunodeficiency virus (FIV), equine infectious anemia virus, and spumavirinae). Between the HIV and SIV, many suitable antigens have been described and can readily be selected. Examples of suitable HIV and SIV antigens include, without limitation the gag, pol, Vif, Vpx, VPR, Env, Tat and Rev proteins, as well as various fragments thereof. In addition, a variety of modifications to these antigens have been described. Suitable antigens for this purpose are known to those of skill in the art. For example, one may select a sequence encoding the gag, pol, Vif, and Vpr, Env, Tat and Rev, amongst other proteins. See, e.g., the modified gag protein which is described in U.S. Pat. No. 5,972,596. See, also, the HIV and SIV proteins described in D.H. Barouch et al, J. Virol., 75(5):2462-2467 (March 2001), and R.R. Amara, et al, Science, 292:69-74 (6 Apr. 2001). These proteins or subunits thereof may be delivered alone, or in combination via separate vectors or from a single vector.

The papovavirus family includes the sub-family polyomaviruses (BKU and JCU viruses) and the sub-family papillomavirus (associated with cancers or malignant progression of papilloma). The adenovirus family includes viruses (EX, AD7, ARD, O.B.) which cause respiratory disease and/or enteritis. The parvovirus family feline parvovirus (feline enteritis), feline panleucopeniavirus, canine parvovirus, and porcine parvovirus. The herpesvirus family includes the sub-family alphaherpesvirinae, which encompasses the genera simplexvirus (HSVI, HSVII), varicellovirus (pseudorabies, varicella zoster) and the sub-family betaherpesvirinae, which includes the genera cytomegalovirus (HCMV, muromegalovirus) and the sub-family gammaherpesvirinae, which includes the genera lymphocryptovirus, EBV (Burkitts lymphoma), infectious rhinotracheitis, Marek's disease virus, and rhadinovirus. The poxvirus family includes the sub-family chordopoxvirinae, which encompasses the genera orthopoxvirus (Variola (Smallpox) and Vaccinia (Cowpox)), parapoxvirus, avipoxvirus, capripoxvirus, leporipoxvirus, suipoxvirus, and the sub-family entomopoxvirinae. The hepadnavirus family includes the Hepatitis B virus. One unclassified virus which may be suitable source of antigens is the Hepatitis delta virus. Still other viral sources may include avian infectious bursal disease virus and porcine respiratory and reproductive syndrome virus. The alphavirus family includes equine arteritis virus and various Encephalitis viruses.

The present invention may also encompass immunogens which are useful to immunize a non-human animal against other pathogens including bacteria, fungi, parasitic microorganisms or multicellular parasites which infect non-human vertebrates, or from a cancer cell or tumor cell. Examples of bacterial pathogens include pathogenic gram-positive cocci include pneumococci; staphylococci; and streptococci. Pathogenic gram-negative cocci include meningococcus; gonococcus. Pathogenic enteric gram-negative bacilli include enterobacteriaceae; pseudomonas, acinetobacteria and eikenella; melioidosis; salmonella; shigella; haemophilus; moraxella; H. ducreyi (which causes chancroid); brucella; Franisella tularensis (which causes tularemia); yersinia (pasteurella); streptobacillus moniliformis and spirillum; Gram-positive bacilli include Listeria monocytogenes; erysipelothrix rhusiopathiae; Corynebacterium diphtheria (diphtheria); cholera; B. anthracis (anthrax); donovanosis (granuloma inguinale); and bartonellosis. Diseases caused by pathogenic anaerobic bacteria include tetanus; botulism; other clostridia; tuberculosis; leprosy; and other mycobacteria. Pathogenic spirochetal diseases include syphilis; treponematoses: yaws, pinta and endemic syphilis; and leptospirosis. Other infections caused by higher pathogen bacteria and pathogenic fungi include actinomycosis; nocardiosis; cryptococcosis, blastomycosis, histoplasmosis and coccidioidomycosis; candidiasis, aspergillosis, and mucormycosis; sporotrichosis; paracoccidiodomycosis, petriellidiosis, torulopsosis, mycetoma and chromomycosis; and dermatophytosis. Rickettsial infections include Typhus fever, Rocky Mountain spotted fever, Q fever, and Rickettsialpox. Examples of mycoplasma and chlamydial infections include: Mycoplasma pneumoniae; lymphogranuloma venereum; psittacosis; and perinatal chlamydial infections. Pathogenic eukaryotes encompass pathogenic protozoans and helminths and infections produced thereby include: amebiasis; malaria; leishmaniasis; trypanosomiasis; toxoplasmosis; Pneumocystis carinii; Trichans; Toxoplasma gondii; babesiosis; giardiasis; trichinosis; filariasis; schistosomiasis; nematodes; trematodes or flukes; and cestode (tapeworm) infections.

Many of these organisms and/or toxins produced thereby have been identified by the Centers for Disease Control [(CDC), Department of Health and Human Services, USA], as agents which have potential for use in biological attacks. For example, some of these biological agents, include, Bacillus anthracis (anthrax), Clostridium botulinum and its toxin (botulism), Yersinia pestis (plague), variola major (smallpox), Francisella tularensis (tularemia), and viral hemorrhagic fever, all of which are currently classified as Category A agents; Coxiella burnetti (Q fever); Brucella species (brucellosis), Burkholderia mallei (glanders), Ricinus communis and its toxin (ricin toxin), Clostridium perfringens and its toxin (epsilon toxin), Staphylococcus species and their toxins (enterotoxin B), all of which are currently classified as Category B agents; and Nipan virus and hantaviruses, which are currently classified as Category C agents. In addition, other organisms, which are so classified or differently classified, may be identified and/or used for such a purpose in the future. It will be readily understood that the viral vectors and other constructs described herein are useful to deliver antigens from these organisms, viruses, their toxins or other by-products, which will prevent and/or treat infection or other adverse reactions with these biological agents.

Administration of the vectors of the invention to deliver immunogens against the variable region of the T cells elicit an immune response including CTLs to eliminate those T cells. In rheumatoid arthritis (RA), several specific variable regions of T cell receptors (TCRs) which are involved in the disease have been characterized. These TCRs include V-3, V-14, V-17 and Vα-17. Thus, delivery of a nucleic acid sequence that encodes at least one of these polypeptides will elicit an immune response that will target T cells involved in RA. In multiple sclerosis (MS), several specific variable regions of TCRs which are involved in the disease have been characterized. These TCRs include V-7 and Vα-10. Thus, delivery of a nucleic acid sequence that encodes at least one of these polypeptides will elicit an immune response that will target T cells involved in MS. In scleroderma, several specific variable regions of TCRs which are involved in the disease have been characterized. These TCRs include V-6, V-8, V-14 and Vα-16, Vα-3C, Vα-7, Vα-14, Vα-15, Vα-16, Vα-28 and Vα-12. Thus, delivery of a nucleic acid molecule that encodes at least one of these polypeptides will elicit an immune response that will target T cells involved in scleroderma.

In one embodiment, the transgene is selected to provide optogenetic therapy. In optogenetic therapy, artificial photoreceptors are constructed by gene delivery of light-activated channels or pumps to surviving cell types in the remaining retinal circuit. This is particularly useful for patients who have lost a significant amount of photoreceptor function, but whose bipolar cell circuitry to ganglion cells and optic nerve remains intact. In one embodiment, the heterologous nucleic acid sequence (transgene) is an opsin. In one embodiment, the opsin is rhodopsin, photopsin, L/M wavelength (red/green)-opsin, or short wavelength (S) opsin (blue). In another embodiment, the opsin is channelrhodopsin or halorhodopsin.

In another embodiment, the transgene is selected for use in gene augmentation therapy, i.e., to provide replacement copy of a gene that is missing or defective. In this embodiment, the transgene may be readily selected by one of skill in the art to provide the necessary replacement gene. In one embodiment, the missing/defective gene is related to an ocular disorder. In another embodiment, the transgene is NYX, GRM6, TRPM1L or GPR179 and the ocular disorder is Congenital Stationary Night Blindness. See, e.g., Zeitz et al, Am J Hum Genet. 2013 Jan. 10; 92(1):67-75. Epub 2012 Dec. 13 which is incorporated herein by reference. In another embodiment, the transgene is RPGR.

In another embodiment, the transgene is selected for use in gene suppression therapy, i.e., expression of one or more native genes is interrupted or suppressed at transcriptional or translational levels. This can be accomplished using short hairpin RNA (shRNA) or other techniques well known in the art. See, e.g., Sun et al, Int J Cancer. 2010 Feb. 1; 126(3):764-74 and O'Reilly M, et al. Am J Hum Genet. 2007 July;81(1):127-35, which are incorporated herein by reference. In this embodiment, the transgene may be readily selected by one of skill in the art based upon the gene which is desired to be silenced.

In another embodiment, the transgene comprises more than one transgene. This may be accomplished using a single vector carrying two or more heterologous sequences, or using two or more vectors each carrying one or more heterologous sequences. In one embodiment, the vector is used for gene suppression (or knockdown) and gene augmentation co-therapy. In knockdown/augmentation co-therapy, the defective copy of the gene of interest is silenced and a non-mutated copy is supplied. In one embodiment, this is accomplished using two or more co-administered vectors. See, Millington-Ward et al, Molecular Therapy, April 2011, 19(4):642-649 which is incorporated herein by reference. The transgenes may be readily selected by one of skill in the art based on the desired result.

In another embodiment, the transgene is selected for use in gene correction therapy. This may be accomplished using, e.g., a zinc-finger nuclease (ZFN)-induced DNA double-strand break in conjunction with an exogenous DNA donor substrate. See, e.g., Ellis et al, Gene Therapy (epub January 2012) 20:35-42 which is incorporated herein by reference. The transgenes may be readily selected by one of skill in the art based on the desired result.

In certain embodiments, the rAAV may be used in gene editing systems, which system may involve one rAAV or co-administration of multiple rAAV stocks. For example, the rAAV may be engineered to deliver SpCas9, SaCas9, ARCUS, Cpf1 (also known as Cas12a), CjCas9, and other suitable gene editing constructs. In certain embodiments, a rAAV-based gene editing nuclease system is provided herein. The gene editing nuclease targets sites in a disease-associated gene, i.e., gene of interest.

In another embodiment, the transgenes useful herein include reporter sequences, which upon expression produce a detectable signal. Such reporter sequences include, without limitation, DNA sequences encoding β-lactamase, β-galactosidase (LacZ), alkaline phosphatase, thymidine kinase, green fluorescent protein (GFP), red fluorescent protein (RFP), chloramphenicol acetyltransferase (CAT), luciferase, membrane bound proteins including, for example, CD2, CD4, CD8, the influenza hemagglutinin protein, and others well known in the art, to which high affinity antibodies directed thereto exist or can be produced by conventional means, and fusion proteins comprising a membrane bound protein appropriately fused to an antigen tag domain from, among others, hemagglutinin or Myc.

These coding sequences, when associated with regulatory elements which drive their expression, provide signals detectable by conventional means, including enzymatic, radiographic, colorimetric, fluorescence or other spectrographic assays, fluorescent activating cell sorting assays and immunological assays, including enzyme linked immunosorbent assay (ELISA), radioimmunoassay (RIA) and immunohistochemistry. For example, where the marker sequence is the LacZ gene, the presence of the vector carrying the signal is detected by assays for beta-galactosidase activity. Where the transgene is green fluorescent protein or luciferase, the vector carrying the signal may be measured visually by color or light production in a luminometer.

Desirably, the transgene encodes a product which is useful in biology and medicine, such as proteins, peptides, RNA, enzymes, or catalytic RNAs. Desirable RNA molecules include shRNA, tRNA, dsRNA, ribosomal RNA, catalytic RNAs, and antisense RNAs. One example of a useful RNA sequence is a sequence which extinguishes expression of a targeted nucleic acid sequence in the treated animal.

The term “heterologous” when used with reference to a protein or a nucleic acid indicates that the protein or the nucleic acid comprises two or more sequences or subsequences which are not found in the same relationship to each other in nature. For instance, the nucleic acid is typically recombinantly produced, having two or more sequences from unrelated genes arranged to make a new functional nucleic acid. For example, in one embodiment, the nucleic acid has a promoter from one gene arranged to direct the expression of a coding sequence from a different gene. Thus, with reference to the coding sequence, the promoter is heterologous.

The term “exogenous” typically is used to refer to two elements which are not from the same source, i.e., of different bacterial or viral origin.

Provided herein are vectors and recombinant AAV which comprise the nucleic acid molecules described herein. In one embodiment, the nucleic acid molecule is comprised within a vector genome. The term “vector genome” when used in the context of an rAAV viral particle refers to the nucleic acid sequences packaged in the rAAV capsid. Typically, vector genomes for rAAV are about 3.5 kb to about 5.2 kb, more preferably about 3.7 kb to 5 kb, or about 4 kb to about 4.7 kb. A vector genome contains AAV ITR sequences at the 5′ terminus and 3′ terminus of the nucleic acid sequences (e.g., expression cassette) to be packaged into the vector.

Such a nucleic acid sequence contains AAV inverted terminal repeat sequences (ITRs). In the examples herein, a vector genome contains, at a minimum, from 5′ to 3′, an AAV 5′ ITR, coding sequence(s), and an AAV 3′ ITR. In certain embodiments, the ITRs are from AAV2, a different source AAV than the capsid, or another full-length ITR may be selected. In certain embodiments, the ITRs are from the same AAV source as the AAV which provides the rep function during production or a trans-complementing AAV. Further, other ITRs may be used. In certain embodiments, the vector genome includes a shortened AAV2 ITR of 130 base pairs, wherein the external A elements is deleted. The shortened ITR is reverted back to the wild type length of 145 base pairs during vector DNA amplification using the internal A element as a template. Further, the vector genome contains an inducible gene expression system which directs expression of the gene products. Suitable components of a vector genome are discussed in more detail herein. The vector genome is sometimes referred to herein as the “minigene”.

The term “recombinant AAV” or “rAAV” as used herein refers to naturally occurring adeno-associated viruses, adeno-associated viruses available to one of skill in the art and/or in light of the composition(s) and method(s) described herein, as well as artificial AAVs. An adeno-associated virus (AAV) viral vector is an AAV Dnase-resistant particle having an AAV protein capsid into which is packaged expression cassette flanked by AAV inverted terminal repeat sequences (ITRs) for delivery to target cells. An AAV capsid is composed of 60 capsid (cap) protein subunits, VP1, VP2, and VP3, that are arranged in an icosahedral symmetry in a ratio of approximately 1:1:10 to 1:1:20, depending upon the selected AAV. Various AAVs may be selected as sources for capsids of AAV viral vectors as identified above. In one embodiment, the AAV capsid is an AAV9 capsid or variant thereof. In certain embodiments, the capsid protein is designated by a number or a combination of numbers and letters following the term “AAV” in the name of the rAAV vector. Unless otherwise specified, the AAV capsid, ITRs, and other selected AAV components described herein, may be readily selected from among any AAV, including, without limitation, the AAVs identified as AAV1, AAV2, AAV3, AAV4, AAV5, AAV6, AAV7, AAV8, AAV9, AAVrh10, AAVhu37, AAVrh32.33, AAV8 bp, AAV7M8 and AAVAnc80, AAV1, AAV2, AAV3, AAV4, AAV5, AAV6, AAV7, AAV8, AAV9.47, AAV9(hu14), AAV10, AAV11, AAV12, AAVrh8, AAVrh74, AAV-DJ8, AAV-DJ, AAVhu68, without limitation. See, e.g., US Published Patent Application No. 2007-0036760-A1; US Published Patent Application No. 2009-0197338-A1; EP 1310571. See also, WO 2003/042397 (AAV7 and other simian AAV), U.S. Pat. Nos. 7,790,449 and 7,282,199 (AAV8), WO 2005/033321 and U.S. Pat. No. 7,906,111 (AAV9), and WO 2006/110689, and WO 2003/042397 (rh.10), WO 2005/033321, WO 2018/160582 (AAVhu68), which are incorporated herein by reference. Other suitable AAVs may include, without limitation, AAVrh90 [PCT/US20/30273, filed Apr. 28, 2020], AAVrh91 [PCT/US20/30266, filed Apr. 28, 2020], AAVrh92, AAVrh93, AAVrh91.93 [PCT/US20/30281, filed Apr. 28, 2020], which are incorporated by reference herein. Other suitable AAV include AAV3B variants which are described in U.S. Provisional Patent Application No. 62/924,112, filed Oct. 21, 2019, and U.S. Provisional Patent Application No. 63/025,753, filed May 15, 2020, describing AAV3B.AR2.01, AAV3B.AR2.02, AAV3B.AR2.03, AAV3B.AR2.04, AAV3B.AR2.05, AAV3B.AR2.06, AAV3B.AR2.07, AAV3B.AR2.08, AAV3B.AR2.10, AAV3B.AR2.11, AAV3B.AR2.12, AAV3B.AR2.13, AAV3B.AR2.14, AAV3B.AR2.15, AAV3B.AR2.16, or AAV3B.AR2.17, which are incorporated herein by reference. These documents also describe other AAV capsids which may be selected for generating rAAV and are incorporated by reference. Among the AAVs isolated or engineered from human or non-human primates (NHP) and well characterized, human AAV2 is the first AAV that was developed as a gene transfer vector; it has been widely used for efficient gene transfer experiments in different target tissues and animal models.

As used herein, relating to AAV, the term “variant” means any AAV sequence which is derived from a known AAV sequence, including those with a conservative amino acid replacement, and those sharing at least 70%, at least 75%, at least 80%, at least 85%, at least 90%, at least 95%, at least 97%, at least 99% or greater sequence identity over the amino acid or nucleic acid sequence. In another embodiment, the AAV capsid includes variants which may include up to about 10% variation from any described or known AAV capsid sequence. That is, the AAV capsid shares about 90% identity to about 99.9% identity, about 95% to about 99% identity or about 97% to about 98% identity to an AAV capsid provided herein and/or known in the art. In one embodiment, the AAV capsid shares at least 95% identity with an AAV capsid. When determining the percent identity of an AAV capsid, the comparison may be made over any of the variable proteins (e.g., vp1, vp2, or vp3).

The term “expression cassette” refers to a nucleic acid molecule which comprises transgene sequences and regulatory sequences therefore (e.g., promoter, enhancer, polyA), which cassette may be packaged into the capsid of a viral vector (e.g., a viral particle). Typically, such an expression cassette for generating a viral vector contains the transgene sequences flanked by packaging signals of the viral genome and other expression control sequences such as those described herein. For example, for an AAV viral vector, the packaging signals are the 5′ inverted terminal repeat (ITR) and the 3′ ITR. In certain embodiments, the term “transgene” may be used interchangeably with “expression cassette”. In other embodiments, the term “transgene” refers solely to the coding sequences for a selected gene.

As described above, the term “about” when used to modify a numerical value means a variation of ±10%, unless otherwise specified.

As used throughout this specification and the claims, the terms “comprise” and “contain” and its variants including, “comprises”, “comprising”, “contains” and “containing”, among other variants, is inclusive of other components, elements, integers, steps and the like. The term “consists of” or “consisting of” are exclusive of other components, elements, integers, steps and the like.

In one embodiment, provided is an rAAV stock comprising rAAV particles having packaged therein a vector genome containing, at a minimum, sequences encoding an activation domain, DNA binding domain comprising a zinc finger homeodomain and one, two or three more FK506 binding protein domain (FKBP) subunit genes, at least 2 to about 12 copies of a zinc finger homeodomain binding site which is a specific binding partner(s) for the zinc finger homeodomain of the DNA binding domain, a mini-promoter and at least one transgene operably linked to the rapamycin regulated expression control sequences. In the presence of an effective amount of a rapamycin or rapalog, transcription of the trangene is induced in a regulatable manner.

As used herein, a “stock” of rAAV refers to a population of rAAV. Despite heterogeneity in their capsid proteins due to deamidation, rAAV in a stock are expected to share an identical vector genome. A stock can include rAAV having capsids with, for example, heterogeneous deamidation patterns characteristic of the selected AAV capsid proteins and a selected production system. The stock may be produced from a single production system or pooled from multiple runs of the production system. A variety of production systems, including but not limited to those described herein, may be selected.

In addition to the regulatable promoter system described herein, the expression cassette and/or vector may contain other appropriate transcription initiation, termination, enhancer sequences, efficient RNA processing signals such as splicing and polyadenylation (polyA) signals; sequences that stabilize cytoplasmic mRNA; sequences that enhance translation efficiency (i.e., Kozak consensus sequence); sequences that enhance protein stability; and when desired, sequences that enhance secretion of the encoded product. Examples of suitable polyA sequences include, e.g., SV40, bovine growth hormone (bGH), and TK polyA. Examples of suitable enhancers include, e.g., CMV enhancer.

These control sequences are “operably linked” to the transgene sequences. As used herein, the term “operably linked” refers to both expression control sequences that are contiguous with the gene of interest and expression control sequences that act in trans or at a distance to control the gene of interest.

In one embodiment, a self-complementary AAV is provided. This viral vector may contain a 45′ ITR and an AAV 3′ ITR. In another embodiment, a single-stranded AAV viral vector is provided. Methods for generating and isolating AAV viral vectors suitable for delivery to a subject are known in the art. See, e.g., U.S. Pat. Nos. 7,790,449; 7,282,199; WO 2003/042397; WO 2005/033321, WO 2006/110689; and U.S. Pat. No. 7,588,772 B2]. In one system, a producer cell line is transiently transfected with a construct that encodes the transgene flanked by ITRs and a construct(s) that encodes rep and cap. In a second system, a packaging cell line that stably supplies rep and cap is transiently transfected with a construct encoding the transgene flanked by ITRs. In each of these systems, AAV virions are produced in response to infection with helper adenovirus or herpesvirus, requiring the separation of the rAAVs from contaminating virus. More recently, systems have been developed that do not require infection with helper virus to recover the AAV—the required helper functions (i.e., adenovirus E1, E2a, VA, and E4 or herpesvirus UL5, UL8, UL52, and UL29, and herpesvirus polymerase) are also supplied, in trans, by the system. In these newer systems, the helper functions can be supplied by transient transfection of the cells with constructs that encode the required helper functions, or the cells can be engineered to stably contain genes encoding the helper functions, the expression of which can be controlled at the transcriptional or posttranscriptional level. In yet another system, the transgene flanked by ITRs and rep/cap genes are introduced into insect cells by infection with baculovirus-based vectors. For reviews on these production systems, see generally, e.g., Zhang et al., 2009, “Adenovirus-adeno-associated virus hybrid for large-scale recombinant adeno-associated virus production,” Human Gene Therapy 20:922-929, the contents of each of which is incorporated herein by reference in its entirety. Methods of making and using these and other AAV production systems are also described in the following US patents, the contents of each of which is incorporated herein by reference in its entirety: U.S. Pat. Nos. 5,139,941; 5,741,683; 6,057,152; 6,204,059; 6,268,213; 6,491,907; 6,660,514; 6,951,753; 7,094,604; 7,172,893; 7,201,898; 7,229,823; and 7,439,065.

A number of suitable purification methods may be selected. Examples of suitable purification methods are described, e.g., in International Patent Publication Nos. WO 2017/100674 (AAV1); WO 2017/100676 (AAV8); WO 2017/100704 (AAVrh10); and WO 2017/160360 (AAV9), which are incorporated by reference herein.

The nucleic acid molecule comprising an inducible gene expression system as described herein may contain at least one internal ribosome binding site, i.e., an IRES, located between the activation domain and the DNA binding domain. Alternatively, the activation domain and the DNA binding domain may be separated by a furin-2a self-cleaving peptide linker [see, e.g., Radcliffe and Mitrophanous, Gene Therapy (2004), 11, 1673-1674].

In one embodiment, the vector genome comprises that of SEQ ID NO: 14 (or nt 184 to 4065), or a sequence sharing at least 90% therewith, wherein the coding sequence for the exemplary rhEPO (nt 4106 to 4684) is replaced with the coding sequence for the desired transgene of interest. In one embodiment, the vector genome comprises that of SEQ ID NO: 22 (or nt 184 to 3907), or a sequence sharing at least 90% therewith, wherein the coding sequence for the exemplary rhEPO (nt 4067-4645) is replaced with the coding sequence for the desired transgene of interest.

In another aspect, a fusion protein comprising one or more of SEQ ID NO: 15, SEQ ID NO: 16, SEQ ID NO: 17, SEQ ID NO: 18, SEQ ID NO: 19, SEQ ID NO: 20, and SEQ ID NO: 21 is provided. Also provided are nucleic acids which encode a fusion protein comprising one or more of SEQ ID NO: 15, SEQ ID NO: 16, SEQ ID NO: 17, SEQ ID NO: 18, SEQ ID NO: 19, SEQ ID NO: 20, and SEQ ID NO: 21. Recombinant AAV encompassing said nucleic acids are also provided.

In another aspect, a fusion protein comprising one or more of SEQ ID NO: 23, SEQ ID NO: 24, SEQ ID NO: 25, SEQ ID NO: 26, SEQ ID NO: 27, SEQ ID NO: 28, and SEQ ID NO: 29 is provided. Also provided are nucleic acids which encode a fusion protein comprising one or more of SEQ ID NO: 23, SEQ ID NO: 24, SEQ ID NO: 25, SEQ ID NO: 26, SEQ ID NO: 27, SEQ ID NO: 28, and SEQ ID NO: 29. Recombinant AAV encompassing said nucleic acids are also provided.

### Uses and Regimens

Suitably, in one embodiment, the compositions are designed to administer an AAV vector carrying the nucleic acid expression cassette encoding the transgene product and regulatory sequences which direct expression of the transgene product thereof in the selected cell.

Following administration of the vector, the inducing agent is used to induce expression of the transgene product construct in vivo. In one embodiment, transgene expression levels may be controlled in a dose-dependent manner by the dose of inducing agent administered to provide a controlled dosage of transgene product.

The compositions can be formulated in dosage units to contain the two or more rAAV, such that each vector stock is present in an amount about 1×10⁹ genome copies (GC) to about 5×10¹³ GC (to treat an average feline subject of about 4 kg in body weight; or an average canine of about 20 kg). In one example, the vector concentration is about 3×10⁹ GC, but other amounts such as about 1×10⁹ GC, about 3×10⁹ GC, about 1×10¹⁰ GC, about 3×10¹⁰ about 1×10¹¹ GC, about 3×10¹¹ GC, about 1×10¹² GC, about 3×10¹² GC, about 1.0×10¹³ GC or about 3.0×10¹³ GC. Suitable concentrations of these vectors may be readily determined based on the desired volume of liquid suspending agent (e.g., in a range of about 250 μL to 100 mL, or higher or lower, depending upon the route of delivery. For example, volumes at the end of the range, or even lower, may be suitable for intranasal delivery, whereas other routes (e.g., systemic delivery) may use higher volumes.

In the case of AAV viral vectors, quantification of the genome copies (“GC”) may be used as the measure of the dose contained in the formulation. Any method known in the art can be used to determine the genome copy (GC) number of the replication-defective virus compositions of the invention. One method for performing AAV GC number titration is as follows: Purified AAV vector samples are first treated with DNase to eliminate un-encapsidated AAV genome DNA or contaminating plasmid DNA from the production process. The nuclease resistant particles are then subjected to heat treatment to release the genome from the capsid. The released genomes are then quantitated by real-time PCR using primer/probe sets targeting specific region of the viral genome (usually poly A signal). Another suitable method for determining genome copies are the quantitative-PCR (qPCR), particularly the optimized qPCR or digital droplet PCR [Lock Martin, et al, Human Gene Therapy Methods. April 2014, 25(2): 115-125. doi:10.1089/hgtb.2013.131, published online ahead of editing Dec. 13, 2013].

The rAAV, preferably suspended in a physiologically compatible carrier, may be administered to a non-human mammalian patient, preferably a canine or feline. Suitable carriers may be readily selected by one of skill in the art in view of the indication for which the transfer virus is directed. For example, one suitable carrier includes saline, which may be formulated with a variety of buffering solutions (e.g., phosphate buffered saline). Other exemplary carriers include sterile saline, lactose, sucrose, maltose, and water. The selection of the carrier is not a limitation of the present invention. Optionally, the compositions of the invention may contain, in addition to the rAAV and carrier(s), other conventional pharmaceutical ingredients, such as preservatives, or chemical stabilizers.

Any suitable route of administration for the vector composition may be selected, including, e.g., systemic, intravenous, intraperitoneal, subcutaneous, intrathecal, intraocular (e.g., intravitreal), or intramuscular administration. In one embodiment, intramuscular administration is utilized.

The term “dimerizer” refers to a compound (e.g., a small molecule, also termed “pharmacologic agent”) that can bind to dimerizer binding domains of the domain fusion proteins and induce dimerization of the fusion proteins. In the constructs described herein, a rapamycin or rapalog is the preferred dimerizer. Any pharmacological agent that dimerizes the domains of the transcription factor, as assayed in vitro can be used. Examples of suitable rapamycins and its analogs, referred to a “rapalogs” are identified earlier in the specification. Any of the dimerizers described in following can be used: US Publication No. 2002/0173474, US Publication No. 2009/0100535, U.S. Pat. Nos. 5,834,266, 7,109,317, 7,485,441, 5,830,462, 5,869,337, 5,871,753, 6,011,018, 6,043,082, 6,046,047, 6,063,625, 6,140,120, 6,165,787, 6,972,193, 6,326,166, 7,008,780, 6,133,456, 6,150,527, 6,506,379, 6,258,823, 6,693,189, 6,127,521, 6,150,137, 6,464,974, 6,509,152, 6,015,709, 6,117,680, 6,479,653, 6,187,757, 6,649,595, 6,984,635, 7,067,526, 7,196,192, 6,476,200, 6,492,106, WO 94118347, WO 96/20951, WO 96/06097, WO 97/31898, WO 96/41865, WO 98/02441, WO 95/33052, WO 99/10508, WO 99/10510, WO 99/36553, WO 99/41258, WO 01114387, ARGENT™ Regulated Transcription Retrovirus Kit, Version 2.0 (9109/02), and ARGENT™ Regulated Transcription Plasmid Kit, Version 2.0 (9/09/02), each of which is incorporated herein by reference in its entirety.

In an embodiment, an amount of pharmaceutical composition comprising a dimerizer of the invention is administered that is in the range of about 0.1 to 5 micrograms (μg)/kilogram (kg). To this end, a pharmaceutical composition comprising a dimerizer of the invention is formulated in doses in the range of about 0.1 mg to about 350 mg to treat to treat an average subject. The amount of pharmaceutical composition comprising a dimerizer of the invention administered is: 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, or 10 mg/kg. The dose of a dimerizer in a formulation is 7, 8, 9, 10, 20, 25, 30, 35, 40, 45, 50, 55, 60, 65, 70, 75, 80, 85 90, 95, 100,125, 150, 175, 200, 225, 250, 275, 300, 325, 350, 375, 400, 400, 425, 450, 475, 500, 525, 550, 575, 600, 625, 650, 675, 700, 725, or 750 mg (to treat an average feline subject of about 4 kg in body weight; or an average canine of about 20 kg). These doses are preferably administered orally. These doses can be given once or repeatedly, such as daily, every other day, weekly, biweekly, or monthly. Preferably, the pharmaceutical compositions are given once weekly for a period of about 4-6 weeks. In some embodiments, a pharmaceutical composition comprising a dimerizer is administered to a subject in one dose, or in two doses, or in three doses, or in four doses, or in five doses, or in six doses or more. The interval between dosages may be determined based the practitioner's determination that there is a need for inhibition of expression of the transgene, for example, in order to ameliorate symptoms caused by expression of the transgene, e.g., toxicity. For example, in some embodiments when the need for transgene ablation is acute, daily dosages of a pharmaceutical composition comprising a dimerizer may be administered. In other embodiments, e.g., when the need for transgene ablation is less acute, or is not acute, weekly dosages of a pharmaceutical composition comprising a dimerizer may be administered.

Pharmaceutical compositions for use as described herein may be formulated in conventional manner using one or more physiologically acceptable carriers or excipients, which may include suspending agents and diluents. The dimerizers and their physiologically acceptable salts and solvates may be formulated for administration by inhalation or insufflation (either through the mouth or the nose) oral, buccal, parenteral, rectal, or transdermal administration. Noninvasive methods of administration are also contemplated.

For oral administration, the pharmaceutical compositions may take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate. Preparations for oral administration may be suitably formulated to give controlled release of the dimerizers.

In another embodiment, the rapamycin (rapalog) is delivered via transdermal patch. Such transdermal patch may be applied for 1 day—several months, and time periods in between.

For buccal administration the compositions may take the form of tablets or lozenges formulated in conventional manner.

For administration by inhalation, the dimerizers for use according to the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g., gelatin for use in an inhaler or insufflator may be formulated containing a powder mix of the dimerizers and a suitable powder base such as lactose or starch.

The dimerizers may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

The dimerizers may also be formulated in rectal compositions such as suppositories or retention enemas, e.g., containing conventional suppository bases such as cocoa butter or other glycerides.

In addition to the formulations described previously, the dimerizers may also be formulated as a depot preparation. Such long acting formulations may be administered by implantation (for example subcutaneously or intramuscularly) or by intramuscular injection. Thus, for example, the dimerizers may be formulated with suitable polymeric or hydrophobic materials (for example as an emulsion in an acceptable oil) or ion exchange resins, or as sparingly soluble derivatives, for example, as a sparingly soluble salt.

The compositions may, if desired, be presented in a pack or dispenser device that may contain one or more unit dosage forms containing the active ingredient. The pack may for example comprise metal or plastic foil, such as a blister pack. The pack or dispenser device may be accompanied by instructions for administration.

Also encompassed is the use of adjuvants in combination with or in admixture with the dimerizers of the invention. Adjuvants contemplated include but are not limited to mineral salt adjuvants or mineral salt gel adjuvants, particulate adjuvants, microparticulate adjuvants, mucosal adjuvants, and immunostimulatory adjuvants. Adjuvants can be administered to a subject as a mixture with dimerizers of the invention, or used in combination with the dimerizers of the invention.

In another embodiment, a composition may contain the rAAV in an amount of about 1.0×10⁹ genome copies (GC)/kilogram (kg) to about 3.0×10¹³ GC/kg, and preferably 1.0×10¹⁰ GC/kg to 3.0×10¹³ GC/kg to a canine or feline patient. Preferably, each rAAV is administered in an amount of about 1.0×10⁸ GC/kg, 5.0×10⁸ GC/kg, 1.0×10⁹ GC/kg, 5.0×10⁹ GC/kg, 1.0×10¹⁰ GC/kg, 5.0×10¹⁰ GC/kg, 1.0×10¹¹ GC/kg, 5.0×10¹¹ GC/kg, or 1.0×10¹² GC/kg, 5.0×10¹² GC/kg, 1.0×10¹³ GC/kg, 3.0×10¹³ GC/kg, 1.0×10¹⁴ GC/kg. In one embodiment, the rAAV is administered in an amount of about 3.0×10¹¹ GC/kg. In one embodiment, the rAAV is administered in an amount of about 3.0×10¹⁰ GC/kg. In one embodiment, the rAAV is administered in an amount of about 3.0×10⁹ GC/kg. In one embodiment, the rAAV is administered in an amount of about 3.0×10⁸ GC/kg.

These doses can be given once or repeatedly, such as daily, every other day, weekly, biweekly, or monthly, or until adequate transgene expression is detected in the patient. In an embodiment, replication-defective virus compositions are given once weekly for a period of about 4-6 weeks, and the mode or site of administration is preferably varied with each administration. Repeated injection is most likely required for complete ablation of transgene expression. The same site may be repeated after a gap of one or more injections. Also, split injections may be given. Thus, for example, half the dose may be given in one site and the other half at another site on the same day.

In one embodiment, the rAAV compositions may be delivered systemically via the liver by injection, e.g., of a mesenteric tributary of portal vein at a dose of about 3.0×10¹² GC/kg. In another embodiment, the rAAV compositions may be delivered systemically via muscle by up to twenty intramuscular injections, e.g., into either the quadriceps or bicep muscles at a dose of about 1.0×10¹⁰ GC/kg to about 3.0×10¹³ GC/kg. In another embodiment, the rAAV compositions may be delivered intracranially, e.g., to the basal forebrain region of the brain containing the nucleus basalis of Meynert (NBM) by bilateral, stereotactic injection, at a dose of about 5.0×10¹¹ GC/kg. In another embodiment, the rAAV compositions may be delivered to the CNS intrathecally, by bilateral intraputaminal and/or intranigral injection at a dose in the range of about 1.0×10¹¹ GC/kg to about 5.0×10¹¹ GC/kg. In another embodiment, the rAAV may be delivered to the joints, e.g., by intra-articular injection at a dose of about 1.0×10¹¹ GC/mL of joint volume for the treatment of inflammatory arthritis. In another embodiment, the rAAV may be delivered to the heart, e.g., by intracoronary infusion injection, at a dose in the range of about 1.4×10¹¹ GC/kg to about 3.0×10¹² GC/kg. In another embodiment, the rAAV compositions may be delivered to the retina, e.g., by injection into the subretinal space at a dose of about 1.5×10¹⁰ GC/kg. In view of this information, other means of delivery to these tissues and organs and other doses can be determined by one of skill in the art.

In one aspect, the invention provides a method for regulating the dose of a pharmacologically active therapeutic product by administering an rAAV vector having a transcription factor under the control of a constitutive or tissue-specific promoter.

The following examples are illustrative only and are not intended to limit the present invention.

### EXAMPLES

### Example 1: In Vitro Expression

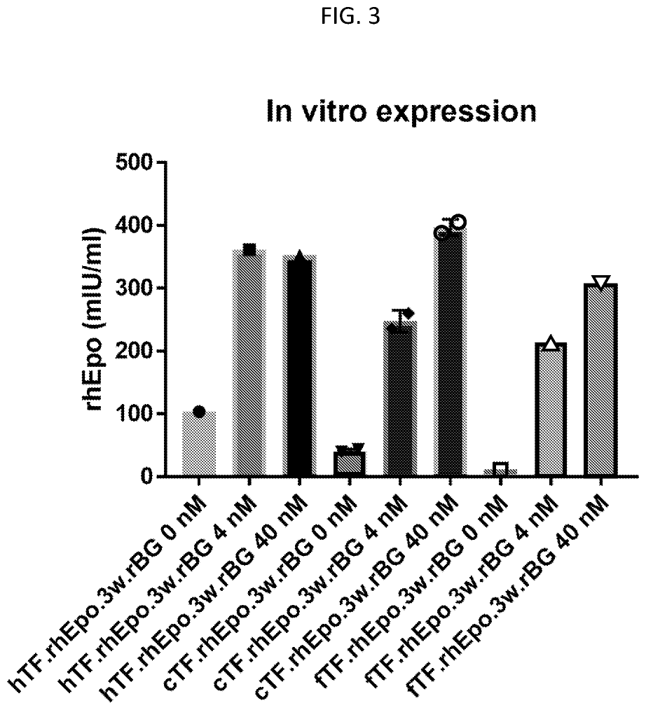

HEK293 cells were transfected with hTF.rhEpo.3w.rBG (human inducible cassette), cTF.rhEpo.3w.rBG (canine inducible cassette), or fTF.rhEpo.3w.rBG (Feline inducible cassette) expressing rhesus macaque Epo (rhEpo). Cells were treated with 0 nM, 4 nM, and 40 nM of rapamycin a day after transfection. Culture supernatants were collected and rhEpo was measured at 48 h after rapamycin treatment. FIG. 3 demonstrates dose dependent expression of rhEPO for feline- and canine-inducible constructs.

(Sequence Listing Free Text)
The following information is provided for sequences
containing free text under numeric identifier <223>.

| SEQ ID NO: (containing | |
|---|---|
| free text) | Free text under <223> |
| 7 | <223> constructed sequence |
| 8 | <223> constructed sequence |
| 9 | <223> constructed sequence |
| 10 | <223> constructed sequence |
| 14 | <223> constructed sequence |
| | <220> |
| | <221> CDS |
| | <222> (856) . . . (1134) |
| | <223> FRE |
| | <220> |
| | <221> CDS |
| | <222> (1183) . . . (1677) |
| | <223> P65 feline |
| | <220> |
| | <221> CDS |
| | <222> (2281) . . . (2316) |
| | <223> Myc NLS |
| | <220> |
| | <221> CDS |
| | <222> (2317) . . . (2685) |
| | <223> ZFHD |
| | <220> |
| | <221> CDS |
| | <222> (2692) . . . (3012) |
| | <223> FKBP |
| | <220> |
| | <221> CDS |
| | <222> (3019) . . . (3339) |
| | <223> FKBPw1 |
| | <220> |
| | <221> CDS |
| | <222> (3346) . . . (3669) |
| | <223> FKBPw2 |
| | <220> |
| | <221> misc_feature |
| | <222> (3802) . . . (3946) |
| 15 | <223> Synthetic Construct |
| 16 | <223> Synthetic Construct |
| 17 | <223> Synthetic Construct |
| 18 | <223> Synthetic Construct |
| 19 | <223> Synthetic Construct |
| 20 | <223> Synthetic Construct |
| 21 | <223> Synthetic Construct |
| 22 | <223> constructed sequence |
| | <220> |
| | <221> CDS |
| | <222> (859) . . . (897) |
| | <223> Myc NLS |
| | <220> |
| | <221> CDS |
| | <222> (898) . . . (1176) |
| | <223> FRB |
| | <220> |
| | <221> CDS |
| | <222> (1183) . . . (1728) |
| | <223> canine P65 TA |
| | <220> |
| | <221> CDS |
| | <222> (2242) . . . (2646) |
| | <223> ZHFD |
| | <220> |
| | <221> CDS |
| | <222> (2653) . . . (2973) |
| | <223> FKBP |
| | <220> |
| | <221> CDS |
| | <222> (2980) . . . (3300) |
| | <223> FKBPw1 |
| | <220> |
| | <221> CDS |
| | <222> (3307) .. (3630) |
| | <223> FKBPw2 |
| | <220> |
| | <221> misc_feature |
| | <222> (3307) . . . (3630) |
| 23 | <223> Synthetic Construct |
| 24 | <223> Synthetic Construct |
| 25 | <223> Synthetic Construct |
| 26 | <223> Synthetic Construct |
| 27 | <223> Synthetic Construct |
| 28 | <223> Synthetic Construct |
| 29 | <223> Synthetic Construct |
| 30 | <223> constructed sequence |
| 31 | <223> constructed sequence |
| 32 | <223> constructed sequence |
| 33 | <223> constructed sequence |
| 34 | <223> constructed sequence |
| 35 | <223> constructed sequence |
| 36 | <223> synthetic construct |
| 37 | <223> synthetic construct |
| 38 | <223> synthetic construct |
| 39 | <223> synthetic construct |

All publications cited in this specification are incorporated herein by reference, as is the Sequence Listing labeled “20-9294PCT_Seq-Listing_ST25.txt”. Also incorporated herein by reference is U.S. Provisional Patent Application No. 63/056,985, filed Jul. 27, 2020. While the invention has been described with reference to particular embodiments, it will be appreciated that modifications can be made without departing from the spirit of the invention. Such modifications are intended to fall within the scope of the appended claims.

1. A nucleic acid molecule comprising an inducible gene expression system, the system comprising:
(a) a transgene encoding a gene product operably linked to expression control sequences comprising a promoter;
(b) an activation domain comprising a canine or feline transactivation domain and a FKBP12-rapamycin binding (FRB) domain of canine or feline FKBP12-rapamycin-associated protein (FRAP);
(c) a DNA binding domain comprising a zinc finger homeodomain (ZFHD) and one, two, or three FK506 binding protein domain (FKBP) subunit genes; and
(d) at least one copy of the binding site for ZFHD followed by a minimal IL2 promoter,
wherein the presence of an effective amount of a rapamycin or a rapalog induces expression of the transgene in a host cell.

2. The nucleic acid molecule according to claim 1, further comprising a 5′ AAV ITR and a 3′ AAV ITR.

3. The nucleic acid molecule of claim 1, wherein the FKBP subunit gene sequences share less than about 85% identity with each other.

4. The nucleic acid molecule according to claim 1, wherein one of the FKBP subunit gene sequences is a native FKBP gene sequence.

5. The nucleic acid molecule according to claim 1, wherein the transactivation domain comprises a portion of NF-κB p65.

6. The nucleic acid molecule according to claim 1, wherein the transactivation domain is a canine p65 sequence.

7. The nucleic acid molecule according to claim 1, wherein the transactivation domain is a feline p65 sequence.

8. The nucleic acid molecule according to claim 1, wherein the promoter is a constitutive promoter.

9. The nucleic acid molecule according to claim 1, wherein the promoter is a tissue specific promoter.

10. The nucleic acid molecule according to claim 1, wherein the promoter is a CMV promoter.

11. The nucleic acid molecule according to claim 1, further comprising an IRES.

12. The nucleic acid molecule according to claim 1, comprising at least 8 copies of the binding site for ZFHD.

13. A nucleic acid molecule comprising: a promoter; an activation domain comprising a canine or feline p65 transactivation domain and a FKBP12-rapamycin binding (FRB) domain of canine or feline FKBP12-rapamycin-associated protein (FRAP); a DNA binding domain comprising a zinc finger homeodomain (ZFHD) and three FK506 binding protein domain (FKBP) subunit genes; 8 copies of the binding site for ZFHD, and a coding sequence for a therapeutic product.

14. The nucleic acid molecule according to claim 1, wherein:
the FRB domain has a sequence sharing at least 90%, 95%, 96%, 96%, 98%, 99% or 100% identity with SEQ ID NO: 1 or SEQ ID NO: 2; and/or
the p65 subunit has a sequence sharing at least 90%, 95%, 96%, 96%, 98%, 99% or 100% identity with SEQ ID NO: 4 or SEQ ID NO: 5.

15. The nucleic acid molecule according to claim 1, wherein the nucleic acid molecule comprises SEQ ID NO: 7, SEQ ID NO: 8, and SEQ ID NO: 9.

16. A recombinant AAV (rAAV) having an AAV capsid, said capsid having packaged therein the nucleic acid molecule according to claim 1.

17. A composition comprising the rAAV according to claim 16 and a pharmaceutically acceptable carrier, excipient, or diluent.

18. A therapeutic regimen for regulating the dose of a therapeutic product, comprising administering the composition of claim 17 to a subject in need thereof, and delivering an effective amount of a rapamycin or a rapalog to induce expression of the therapeutic product in a host cell of the subject.

19-26. (canceled)

27. The nucleic acid molecule according to claim 1, wherein the ZFHD homeodomain sequence is SEQ ID NO: 30.

28. The nucleic acid molecule according to claim 1, wherein the FKBP subunit gene sequences are SEQ ID NO: 7, SEQ ID NO: 8, and SEQ ID NO: 9.

29. The nucleic acid molecule according to claim 1, wherein the 8XZFHD sequence is SEQ ID NO: 31.

30. The nucleic acid molecule according to claim 1, wherein the IL2 minimal promoter sequence is SEQ ID NO: 11 or SEQ ID NO: 12.

31-32. (canceled)


SEQUENCE LISTING (39 sequences)

SEQ ID NO: 1 - DNA, 279 bp, Canis familiaris
atcctctggcatgagatgtggcatgaaggcctggaagaggcatctcgtttgtactttggggaaaggaacgtgaaaggcatgtttgaggtgctggagcccttgcatgctatgatggaacggggcccccagactctgaaggaaacatcctttaatcaggcctatggtcgagatttaatggaggcccaagagtggtgcaggaagtacatgaaatcagggaacgtcaaggacctcacccaagcctgggacctctattatcacgtgttccgacgaatctcaaag

SEQ ID NO: 2 - DNA, 279 bp, Felis catus
atggactatcctgctgccaagagggtcaagttggactctagaatcctctggcatgagatgtggcatgaaggcctggaagaggcatctcgtttgtactttggggaaaggaacgtgaaaggcatgtttgaggtgctggagcccttgcatgctatgatggaacggggcccccagactctgaaggaaacatcctttaatcaggcctatggtcgagatttaatggaggcccaagagtggtgcaggaagtacatgaaatcagggaacgtcaaggacctcacccaa

SEQ ID NO: 3 - DNA, 279 bp, Homo sapiens
atggactatcctgctgccaagagggtcaagttggactctagaatcctctggcatgagatgtggcatgaaggcctggaagaggcatctcgtttgtactttggggaaaggaacgtgaaaggcatgtttgaggtgctggagcccttgcatgctatgatggaacggggcccccagactctgaaggaaacatcctttaatcaggcctatggtcgagatttaatggaggcccaagagtggtgcaggaagtacatgaaatcagggaatgtcaaggacctcctccaa

SEQ ID NO: 4 - DNA, 546 bp, Canis familiaris
gaggaattcagccccatggtgttctctagcggccagatcagctctcagacaagcgcccttgcttctgcccctgctcctgctccaattctggctccagctcctcctgcaccagctccagcacctattctggcacctggactggctcaggctatggctccaccagcaccaaagacaacacaggccggcgagggaacactgacagaagctctgctgcagctccagttcgacgccgacgaggatctgggagccctgctgggcaattctgctgatcctgccgtgttcaccgatctggccagcgtggacaatagcgagttccagcagctcctgaaccagggcgttagtgtggcccctcatacagccgagcctatgctgatggaataccccgaggctatcaccagactggtcaccggctctcaaagacctccagatcctgtgcctgctccagtgggagcatctgggctccccaatggcctcctttcaggagatgaagacttctcctccattgcggacatggacttctcagccctgctgagtcagatcagctcc

SEQ ID NO: 5 - DNA, 495 bp, Felis catus
gaggaattcagccccatggtgttccccagcggacagatcccatctcagacaccagctctggctccagctcctgctcctattcttgctcctgcaccagcctctgtgcctgctccagcacctgttccggcaccagcaccagctccggctccggctccagcaccaatacttgctccaggactggctcaggctgtggtgccgccagctcctaaaacaacacaagccggcgagggcacactgacagaagctctgctgcatctgcagttcgacgccgacgaggatctgggagccctgctgggcaattctgctgatcctgccgtgttcaccgatctggccagcgtggacaatagcgagttccagcagctcctgaaccagggcgttagtgtggctcctcatcctgccgagcctatgctgatggaataccccgaggctatcaccagactggtcaccggctctcaaagacctccagatccagcaccggcaccactgggagcatct

SEQ ID NO: 6 - DNA, 573 bp, Homo sapiens
gatgagtttcccaccatggtgtttccttctgggcagatcagccaggcctcggccttggccccggcccctccccaagtcctgccccaggctccagcccctgcccctgctccagccatggtatcagctctggcccaggccccagcccctgtcccagtcctagccccaggccctcctcaggctgtggccccacctgcccccaagcccacccaggctggggaaggaacgctgtcagaggccctgctgcagctgcagtttgatgatgaagacctgggggccttgcttggcaacagcacagacccagctgtgttcacagacctggcatccgtcgacaactccgagtttcagcagctgctgaaccagggcatacctgtggccccccacacaactgagcccatgctgatggagtaccctgaggctataactcgcctagtgacaggggcccagaggccccccgacccagctcctgctccactgggggccccggggctccccaatggcctcctttcaggagatgaagacttctcctccattgcggacatggacttctcagccctgctgagtcagatcagctcc

SEQ ID NO: 7 - DNA, 321 bp, Artificial Sequence
  constructed sequence
ggagtgcaggtggaaaccatctccccaggagacgggcgcaccttccccaagcgcggccagacctgcgtggtgcactacaccgggatgcttgaagatggaaagaaatttgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccaaacgccactctcgtcttcgatgtggagcttctaaaactggaa

SEQ ID NO: 8 - DNA, 321 bp, Artificial Sequence
  constructed sequence
ggcgttcaggtggaaaccatcagtccaggggatggccgaacttttccaaagagagggcagacttgcgtcgtgcattatactggtatgctggaggatgggaaaaagttcgactcttccagagatcggaacaaaccattcaaattcatgctcgggaaacaggaagttatccgcggatgggaggagggcgtggcccagatgtccgtgggccagcgcgccaagctaaccatctccccagactacgcctacggagccaccggacaccccggtatcatacccccaaacgccacccttgtgtttgacgtggaactgcttaagctagag

SEQ ID NO: 9 - DNA, 324 bp, Artificial Sequence
  constructed sequence
ggcgtgcaggtcgagaccatcagccccggcgacggccgcacctttcccaagagaggccagacttgcgtggtccactacaccggcatgctggaggacggcaagaagttcgacagcagccgcgaccgcaacaagcccttcaagttcatgctgggcaaacaggaagtgatccgcggctgggaggaaggcgtggctcagatgagcgtggggcagcgggccaagctgaccatcagccccgactatgcctacggcgccaccggccaccccggcatcatcccccccaacgccaccctcgtgttcgacgtggagctgctgaagctggagtga

SEQ ID NO: 10 - DNA, 978 bp, Artificial Sequence
  constructed sequence
ggagtgcaggtggaaaccatctccccaggagacgggcgcaccttccccaagcgcggccagacctgcgtggtgcactacaccgggatgcttgaagatggaaagaaatttgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccaaacgccactctcgtcttcgatgtggagcttctaaaactggaaactagaggcgttcaggtggaaaccatcagtccaggggatggccgaacttttccaaagagagggcagacttgcgtcgtgcattatactggtatgctggaggatgggaaaaagttcgactcttccagagatcggaacaaaccattcaaattcatgctcgggaaacaggaagttatccgcggatgggaggagggcgtggcccagatgtccgtgggccagcgcgccaagctaaccatctccccagactacgcctacggagccaccggacaccccggtatcatacccccaaacgccacccttgtgtttgacgtggaactgcttaagctagagactagaggcgtgcaggtcgagaccatcagccccggcgacggccgcacctttcccaagagaggccagacttgcgtggtccactacaccggcatgctggaggacggcaagaagttcgacagcagccgcgaccgcaacaagcccttcaagttcatgctgggcaaacaggaagtgatccgcggctgggaggaaggcgtggctcagatgagcgtggggcagcgggccaagctgaccatcagccccgactatgcctacggcgccaccggccaccccggcatcatcccccccaacgccaccctcgtgttcgacgtggagctgctgaagctggagtga

SEQ ID NO: 11 - DNA, 121 bp, Homo sapiens
aacattttgacacccccataatatttttccagaattaacagtataaattgcatctcttgttcaagagttccctatcactctctttaatcactactcacagtaacctcaactcctgccacaa

SEQ ID NO: 12 - DNA, 107 bp, Homo sapiens
aacattttgacacccccataatatttttccagaattaacagtataaattgcatctcttgttcaagagttccctatcactctctttaatcactactcacagtaacctc

SEQ ID NO: 13 - DNA, 639 bp, Human cytomegalovirus
acgcgttcgagctcgccccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgggggatcttggtggcgtgaaactcccg

SEQ ID NO: 14 - DNA, 4949 bp, Artificial Sequence
  constructed sequence
  CDS (856)..(1134): FRE
  CDS (1183)..(1677): P65 feline
  CDS (2281)..(2316): Myc NLS
  CDS (2317)..(2685): ZFHD
  CDS (2692)..(3012): FKBP
  CDS (3019)..(3339): FKBPw1
  CDS (3346)..(3669): FKBPw2
  misc_feature (3802)..(3946)
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctagtacgcgttcgagctcgccccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgggggatcttggtggcgtgaaactcccgcagatcttcggccagcgaattccagaagccaccatggactatcctgctgccaagagggtcaagttggactctagaatcctctggcatgagatgtggcatgaaggcctggaagaggcatctcgtttgtactttggggaaaggaacgtgaaaggcatgtttgaggtgctggagcccttgcatgctatgatggaacggggcccccagactctgaaggaaacatcctttaatcaggcctatggtcgagatttaatggaggcccaagagtggtgcaggaagtacatgaaatcagggaacgtcaaggacctcacccaagcctgggacctctattatcacgtgttccgacgaatctcaaagactagagaggaattcagccccatggtgttccccagcggacagatcccatctcagacaccagctctggctccagctcctgctcctattcttgctcctgcaccagcctctgtgcctgctccagcacctgttccggcaccagcaccagctccggctccggctccagcaccaatacttgctccaggactggctcaggctgtggtgccgccagctcctaaaacaacacaagccggcgagggcacactgacagaagctctgctgcatctgcagttcgacgccgacgaggatctgggagccctgctgggcaattctgctgatcctgccgtgttcaccgatctggccagcgtggacaatagcgagttccagcagctcctgaaccagggcgttagtgtggctcctcatcctgccgagcctatgctgatggaataccccgaggctatcaccagactggtcaccggctctcaaagacctccagatccagcaccggcaccactgggagcatctgggctccccaatggcctcctttcaggagatgaagacttctcctccattgcggacatggacttctcagccctgctgagtcagatcagctcctgataattccggttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataataccatggactatcctgctgccaagagggtcaagttggactctagagaacgcccatatgcttgccctgtcgagtcctgcgatcgccgcttttctcgctcggatgagcttacccgccatatccgcatccacacaggccagaagcccttccagtgtcgaatctgcatgcgtaacttcagtcgtagtgaccaccttaccacccacatccgcacccacacaggcggcggccgcaggaggaagaaacgcaccagcatagagaccaacatccgtgtggccttagagaagagtttcttggagaatcaaaagcctacctcggaagagatcactatgattgctgatcagctctccatggaaaaagaggtgattcgtgtttggttctgtaaccgccgccagaaagaaaaaagaatcaacactagaggagtgcaggtggaaaccatctccccaggagacgggcgcaccttccccaagcgcggccagacctgcgtggtgcactacaccgggatgcttgaagatggaaagaaatttgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccaaacgccactctcgtcttcgatgtggagcttctaaaactggaaactagaggcgttcaggtggaaaccatcagtccaggggatggccgaacttttccaaagagagggcagacttgcgtcgtgcattatactggtatgctggaggatgggaaaaagttcgactcttccagagatcggaacaaaccattcaaattcatgctcgggaaacaggaagttatccgcggatgggaggagggcgtggcccagatgtccgtgggccagcgcgccaagctaaccatctccccagactacgcctacggagccaccggacaccccggtatcatacccccaaacgccacccttgtgtttgacgtggaactgcttaagctagagactagaggcgtgcaggtcgagaccatcagccccggcgacggccgcacctttcccaagagaggccagacttgcgtggtccactacaccggcatgctggaggacggcaagaagttcgacagcagccgcgaccgcaacaagcccttcaagttcatgctgggcaaacaggaagtgatccgcggctgggaggaaggcgtggctcagatgagcgtggggcagcgggccaagctgaccatcagccccgactatgcctacggcgccaccggccaccccggcatcatcccccccaacgccaccctcgtgttcgacgtggagctgctgaagctggagtgataaggatccccactccagtgcccaccagccttgtcctaataaaattaagttgcatcattttgtctgactaggtgtccttctataatattatggggtggaggggggtggtttggagcaaggcgcgtgctagctaatgatgggcgctcgagtaatgatgggcggtcgactaatgatgggcgctcgagtaatgatgggcgtctagctaatgatgggcgctcgagtaatgatgggcggtcgactaatgatgggcgctcgagtaatgatgggcgtctagaacgcgaattaattcaacattttgacacccccataatatttttccagaattaacagtataaattgcatctcttgttcaagagttccctatcactctctttaatcactactcacagtaacctcaactcctgccacaagcttgccctgcagcgggaattccaccatgggggtgcacgaatgtcctgcctggctgtggcttctcctgtctctgctctcgctccctctgggcctcccagtcccgggcgccccaccacgcctcatctgtgacagccgagtcctggagaggtacctcttggaggccaaggaggccgagaatgtcacgatgggctgttccgaaagctgcagcttgaatgagaatatcaccgtcccagacaccaaagttaacttctatgcctggaagaggatggaggtcgggcagcaggctgtagaagtctggcagggcctggccctgctctcagaagctgtcctgcggggccaggccgtgttggccaactcttcccagcctttcgagcccctgcagctgcacatggataaagccatcagtggccttcgcagcatcaccactctgcttcgggcgctgggagcccaggaagccatctccctcccagatgcggcctcggctgctccactccgaaccatcactgctgacactttctgcaaactcttccgagtctactccaatttcctccggggaaagctgaagctgtacacgggggaggcctgcaggagaggggacagatgaccatcgattacgcctaataaagagctcagatgcatgcatcagagtgtgttggttttttgtgtgtggcgcgccgatcctcgagactagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcca

SEQ ID NO: 15 - protein, 93 aa, Artificial Sequence
  Synthetic Construct
MDYPAAKRVKLDSRILWHEMWHEGLEEASRLYFGERNVKGMFEVLEPLHAMMERGPQTLKETSFNQAYGRDLMEAQEWCRKYMKSGNVKDLTQ

SEQ ID NO: 16 - protein, 165 aa, Artificial Sequence
  Synthetic Construct
EEFSPMVFPSGQIPSQTPALAPAPAPILAPAPASVPAPAPVPAPAPAPAPAPAPILAPGLAQAVVPPAPKTTQAGEGTLTEALLHLQFDADEDLGALLGNSADPAVFTDLASVDNSEFQQLLNQGVSVAPHPAEPMLMEYPEAITRLVTGSQRPPDPAPAPLGAS

SEQ ID NO: 17 - protein, 12 aa, Artificial Sequence
  Synthetic Construct
MDYPAAKRVKLD

SEQ ID NO: 18 - protein, 123 aa, Artificial Sequence
  Synthetic Construct
SRERPYACPVESCDRRFSRSDELTRHIRIHTGQKPFQCRICMRNFSRSDHLTTHIRTHTGGGRRRKKRTSIETNIRVALEKSFLENQKPTSEEITMIADQLSMEKEVIRVWFCNRRQKEKRIN

SEQ ID NO: 19 - protein, 107 aa, Artificial Sequence
  Synthetic Construct
GVQVETISPGDGRTFPKRGQTCVVHYTGMLEDGKKFDSSRDRNKPFKFMLGKQEVIRGWEEGVAQMSVGQRAKLTISPDYAYGATGHPGIIPPNATLVFDVELLKLE

SEQ ID NO: 20 - protein, 107 aa, Artificial Sequence; identical to SEQ ID NO: 19
  Synthetic Construct

SEQ ID NO: 21 - protein, 107 aa, Artificial Sequence; identical to SEQ ID NO: 19
  Synthetic Construct

SEQ ID NO: 22 - DNA, 4910 bp, Artificial Sequence
  constructed sequence
  CDS (859)..(897): Myc NLS
  CDS (898)..(1176): FRB
  CDS (1183)..(1728): canine P65 TA
  CDS (2242)..(2646): ZHFD
  CDS (2653)..(2973): FKBP
  CDS (2980)..(3300): FKBPw1
  CDS (3307)..(3630): FKBPw2
  misc_feature (3307)..(3630)
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctagtacgcgttcgagctcgccccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgggggatcttggtggcgtgaaactcccgcagatcttcggccagcgaattccagaagccaccatggactatcctgctgccaagagggccagattggactctagaatcctctggcatgagatgtggcatgaaggcctggaagaggcatctcgtttgtactttggggaaaggaacgtgaaaggcatgtttgaggtgctggagcccttgcatgctatgatggaacggggcccccagactctgaaggaaacatcctttaatcaggcctatggtcgagatttaatggaggcccaagagtggtgcaggaagtacatgaaatcagggaacgtcaaggacctcacccaagcctgggacctctattatcacgtgttccgacgaatctcaaagactagagaggaattcagccccatggtgttctctagcggccagatcagctctcagacaagcgcccttgcttctgcccctgctcctgctccaattctggctccagctcctcctgcaccagctccagcacctattctggcacctggactggctcaggctatggctccaccagcaccaaagacaacacaggccggcgagggaacactgacagaagctctgctgcagctccagttcgacgccgacgaggatctgggagccctgctgggcaattctgctgatcctgccgtgttcaccgatctggccagcgtggacaatagcgagttccagcagctcctgaaccagggcgttagtgtggcccctcatacagccgagcctatgctgatggaataccccgaggctatcaccagactggtcaccggctctcaaagacctccagatcctgtgcctgctccagtgggagcatctgggctccccaatggcctcctttcaggagatgaagacttctcctccattgcggacatggacttctcagccctgctgagtcagatcagctcctgataattccggttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataataccatggactatcctgctgccaagagggccagattggactctagagaacgcccatatgcttgccctgtcgagtcctgcgatcgccgcttttctcgctcggatgagcttacccgccatatccgcatccacacaggccagaagcccttccagtgtcgaatctgcatgcgtaacttcagtcgtagtgaccaccttaccacccacatccgcacccacacaggcggcggccgcaggaggaagaaacgcaccagcatagagaccaacatccgtgtggccttagagaagagtttcttggagaatcaaaagcctacctcggaagagatcactatgattgctgatcagctctccatggaaaaagaggtgattcgtgtttggttctgtaaccgccgccagaaagaaaaaagaatcaacactagaggagtgcaggtggaaaccatctccccaggagacgggcgcaccttccccaagcgcggccagacctgcgtggtgcactacaccgggatgcttgaagatggaaagaaatttgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccaaacgccactctcgtcttcgatgtggagcttctaaaactggaaactagaggcgttcaggtggaaaccatcagtccaggggatggccgaacttttccaaagagagggcagacttgcgtcgtgcattatactggtatgctggaggatgggaaaaagttcgactcttccagagatcggaacaaaccattcaaattcatgctcgggaaacaggaagttatccgcggatgggaggagggcgtggcccagatgtccgtgggccagcgcgccaagctaaccatctccccagactacgcctacggagccaccggacaccccggtatcatacccccaaacgccacccttgtgtttgacgtggaactgcttaagctagagactagaggcgtgcaggtcgagaccatcagccccggcgacggccgcacctttcccaagagaggccagacttgcgtggtccactacaccggcatgctggaggacggcaagaagttcgacagcagccgcgaccgcaacaagcccttcaagttcatgctgggcaaacaggaagtgatccgcggctgggaggaaggcgtggctcagatgagcgtggggcagcgggccaagctgaccatcagccccgactatgcctacggcgccaccggccaccccggcatcatcccccccaacgccaccctcgtgttcgacgtggagctgctgaagctggagtgataaggatccccactccagtgcccaccagccttgtcctaataaaattaagttgcatcattttgtctgactaggtgtccttctataatattatggggtggaggggggtggtttggagcaaggcgcgtgctagctaatgatgggcgctcgagtaatgatgggcggtcgactaatgatgggcgctcgagtaatgatgggcgtctagctaatgatgggcgctcgagtaatgatgggcggtcgactaatgatgggcgctcgagtaatgatgggcgtctagaacgcgaattaattcaacattttgacacccccataatatttttccagaattaacagtataaattgcatctcttgttcaagagttccctatcactctctttaatcactactcacagtaacctcaactcctgccacaagcttgccctgcagcgggaattccaccatgggggtgcacgaatgtcctgcctggctgtggcttctcctgtctctgctctcgctccctctgggcctcccagtcccgggcgccccaccacgcctcatctgtgacagccgagtcctggagaggtacctcttggaggccaaggaggccgagaatgtcacgatgggctgttccgaaagctgcagcttgaatgagaatatcaccgtcccagacaccaaagttaacttctatgcctggaagaggatggaggtcgggcagcaggctgtagaagtctggcagggcctggccctgctctcagaagctgtcctgcggggccaggccgtgttggccaactcttcccagcctttcgagcccctgcagctgcacatggataaagccatcagtggccttcgcagcatcaccactctgcttcgggcgctgggagcccaggaagccatctccctcccagatgcggcctcggctgctccactccgaaccatcactgctgacactttctgcaaactcttccgagtctactccaatttcctccggggaaagctgaagctgtacacgggggaggcctgcaggagaggggacagatgaccatcgattacgcctaataaagagctcagatgcatgcatcagagtgtgttggttttttgtgtgtggcgcgccgatcctcgagactagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcca

SEQ ID NO: 23 - protein, 13 aa, Artificial Sequence
  Synthetic Construct
DYPAAKRARLDSR

SEQ ID NO: 24 - protein, 93 aa, Artificial Sequence [sequence omitted]

SEQ ID NO: 25 - protein, 182 aa, Artificial Sequence
  Synthetic Construct
EEFSPMVFSSGQISSQTSALASAPAPAPILAPAPPAPAPAPILAPGLAQAMAPPAPKTTQAGEGTLTEALLQLQFDADEDLGALLGNSADPAVFTDLASVDNSEFQQLLNQGVSVAPHTAEPMLMEYPEAITRLVTGSQRPPDPVPAPVGASGLPNGLLSGDEDFSSIADMDFSALLSQISS

SEQ ID NO: 26 - protein, 135 aa, Artificial Sequence
  Synthetic Construct
MDYPAAKRARLDSRERPYACPVESCDRRFSRSDELTRHIRIHTGQKPFQCRICMRNFSRSDHLTTHIRTHTGGGRRRKKRTSIETNIRVALEKSFLENQKPTSEEITMIADQLSMEKEVIRVWFCNRRQKEKRIN

SEQ ID NO: 27 - protein, 107 aa, Artificial Sequence; identical to SEQ ID NO: 19
  Synthetic Construct

SEQ ID NO: 28 - protein, 107 aa, Artificial Sequence; identical to SEQ ID NO: 19
  Synthetic Construct

SEQ ID NO: 29 - protein, 107 aa, Artificial Sequence; identical to SEQ ID NO: 19
  Synthetic Construct

SEQ ID NO: 30 - DNA, 405 bp, Artificial Sequence
  constructed sequence
atggactatcctgctgccaagagggccagattggactctagagaacgcccatatgcttgccctgtcgagtcctgcgatcgccgcttttctcgctcggatgagcttacccgccatatccgcatccacacaggccagaagcccttccagtgtcgaatctgcatgcgtaacttcagtcgtagtgaccaccttaccacccacatccgcacccacacaggcggcggccgcaggaggaagaaacgcaccagcatagagaccaacatccgtgtggccttagagaagagtttcttggagaatcaaaagcctacctcggaagagatcactatgattgctgatcagctctccatggaaaaagaggtgattcgtgtttggttctgtaaccgccgccagaaagaaaaaagaatcaac

SEQ ID NO: 31 - DNA, 145 bp, Artificial Sequence
  constructed sequence
aatgatgggcgctcgagtaatgatgggcggtcgactaatgatgggcgctcgagtaatgatgggcgtctagctaatgatgggcgctcgagtaatgatgggcggtcgactaatgatgggcgctcgagtaatgatgggcgtctagaac

SEQ ID NO: 32 - DNA, 18 bp, Artificial Sequence
  constructed sequence
aatgatgggcgctcgagt

SEQ ID NO: 33 - DNA, 18 bp, Artificial Sequence
  constructed sequence
aatgatgggcggtcgact

SEQ ID NO: 34 - DNA, 18 bp, Artificial Sequence
  constructed sequence
aatgatgggcgtctagct

SEQ ID NO: 35 - DNA, 19 bp, Artificial Sequence
  constructed sequence
aatgatgggcgtctagaac

SEQ ID NO: 36 - protein, 304 aa, Artificial Sequence
  synthetic construct
MDYPAAKRVKLDSRILWHEMWHEGLEEASRLYFGERNVKGMFEVLEPLHAMMERGPQTLKETSFNQAYGRDLMEAQEWCRKYMKSGNVKDLTQAWDLYYHVFRRISKTREEFSPMVFPSGQIPSQTPALAPAPAPILAPAPASVPAPAPVPAPAPAPAPAPAPILAPGLAQAVVPPAPKTTQAGEGTLTEALLHLQFDADEDLGALLGNSADPAVFTDLASVDNSEFQQLLNQGVSVAPHPAEPMLMEYPEAITRLVTGSQRPPDPAPAPLGASGLPNGLLSGDEDFSSIADMDFSALLSQISS

SEQ ID NO: 37 - protein, 462 aa, Artificial Sequence
  synthetic construct
MDYPAAKRVKLDSRERPYACPVESCDRRFSRSDELTRHIRIHTGQKPFQCRICMRNFSRSDHLTTHIRTHTGGGRRRKKRTSIETNIRVALEKSFLENQKPTSEEITMIADQLSMEKEVIRVWFCNRRQKEKRINTRGVQVETISPGDGRTFPKRGQTCVVHYTGMLEDGKKFDSSRDRNKPFKFMLGKQEVIRGWEEGVAQMSVGQRAKLTISPDYAYGATGHPGIIPPNATLVFDVELLKLETRGVQVETISPGDGRTFPKRGQTCVVHYTGMLEDGKKFDSSRDRNKPFKFMLGKQEVIRGWEEGVAQMSVGQRAKLTISPDYAYGATGHPGIIPPNATLVFDVELLKLETRGVQVETISPGDGRTFPKRGQTCVVHYTGMLEDGKKFDSSRDRNKPFKFMLGKQEVIRGWEEGVAQMSVGQRAKLTISPDYAYGATGHPGIIPPNATLVFDVELLKLE

SEQ ID NO: 38 - protein, 291 aa, Artificial Sequence
  synthetic construct
MDYPAAKRARLDSRILWHEMWHEGLEEASRLYFGERNVKGMFEVLEPLHAMMERGPQTLKETSFNQAYGRDLMEAQEWCRKYMKSGNVKDLTQAWDLYYHVFRRISKTREEFSPMVFSSGQISSQTSALASAPAPAPILAPAPPAPAPAPILAPGLAQAMAPPAPKTTQAGEGTLTEALLQLQFDADEDLGALLGNSADPAVFTDLASVDNSEFQQLLNQGVSVAPHTAEPMLMEYPEAITRLVTGSQRPPDPVPAPVGASGLPNGLLSGDEDFSSIADMDFSALLSQISS

SEQ ID NO: 39 - protein, 462 aa, Artificial Sequence
  synthetic construct
MDYPAAKRARLDSRERPYACPVESCDRRFSRSDELTRHIRIHTGQKPFQCRICMRNFSRSDHLTTHIRTHTGGGRRRKKRTSIETNIRVALEKSFLENQKPTSEEITMIADQLSMEKEVIRVWFCNRRQKEKRINTRGVQVETISPGDGRTFPKRGQTCVVHYTGMLEDGKKFDSSRDRNKPFKFMLGKQEVIRGWEEGVAQMSVGQRAKLTISPDYAYGATGHPGIIPPNATLVFDVELLKLETRGVQVETISPGDGRTFPKRGQTCVVHYTGMLEDGKKFDSSRDRNKPFKFMLGKQEVIRGWEEGVAQMSVGQRAKLTISPDYAYGATGHPGIIPPNATLVFDVELLKLETRGVQVETISPGDGRTFPKRGQTCVVHYTGMLEDGKKFDSSRDRNKPFKFMLGKQEVIRGWEEGVAQMSVGQRAKLTISPDYAYGATGHPGIIPPNATLVFDVELLKLE